(19) United States
(12) Patent Application Publication
(10) Pub. No.: US 2024/0024502 A1
(43) Pub. Date: Jan. 25, 2024

(54) ANTI-MSLN BINDING AGENTS, CONJUGATES THEREOF AND METHODS OF USING THE SAME

(71) Applicant: Ardeagen Corporation, Woodinville, WA (US)

(72) Inventors: May Kung SUTHERLAND, Bothell, WA (US); Maria Leia SMITH, Seattle, WA (US)

(21) Appl. No.: 18/247,627

(22) PCT Filed: Oct. 15, 2021
(86) PCT No.: PCT/US2021/055313

§ 371 Date: Mar. 31, 2023

Related U.S. Application Data
(60) Provisional application No. 63/093,254, filed on Oct. 18, 2020.

(51) Int. Cl. A61K 47/68; A61K 45/06; C07K 16/28; C07K 16/30; A61P 35/00

(52) U.S. Cl. CPC A61K 47/6851; A61K 45/06; C07K 16/2818; C07K 16/30; C07K 16/2827; A61P 35/00

(57) ABSTRACT

The present disclosure provides anti-MSLN antibodies, antigen binding portions thereof and MSLN conjugates thereof for use in the treatment of cancer.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 120301_402WO_SEQUENCE_LISTING.txt. The text file is 31.4 KB, was created on Oct. 14, 2021, and is being submitted electronically via EFS-Web.

### BACKGROUND

Effective and tumor-targeted treatment for various types of cancer remain an important need for improvement of survival for patients. Worldwide in 2018, there were 2.1 million cases of lung cancer with 1.7 million deaths. In the same year, gastric and colorectal cancers accounted for 783,000 deaths and 881,000 deaths, respectively. Currently, there are few therapeutic options for pancreatic and esophageal cancers. Mesothelin or MSLN, is overexpressed on human malignant cells, and is known to be highly expressed in these tumors, as well as in mesothelioma, triple negative breast cancer, ovarian adenocarcinoma, uterine serous carcinoma, cholangiocarcinoma, endometrial adenocarcinoma, soft tissue carcinomas and head and neck cancers. With the limited expression of MSLN in normal adult tissues, targeting of MSLN using an antibody armed with a cytotoxic agent (antibody-drug conjugate) provides a way of selectively attacking the cancer cells and sparing normal tissues.

### BRIEF SUMMARY

The present disclosure provides in part on ARD110 Mesothelin (MSLN) binding antibodies, antigen-binding portions thereof and related binding agents that specifically bind to MSLN, as well as conjugates thereof, that exhibit improved therapeutic properties MSLN is an important and advantageous therapeutic target for the treatment of certain cancers. The MSLN-binding antibodies, antigen binding portions thereof and binding agents and conjugates thereof provide compositions and methods based on the use of such antibodies, antigen binding portions and related binding agents, and conjugates thereof, in the treatment of MSLN+ cancers. Accordingly, the present disclosure provides methods, compositions, kits, and articles of manufacture related to ARD110 anti-MSLN antibodies, antigen-binding portions, binding agents and conjugates.

In some embodiments, a conjugate is provided comprising: a binding agent comprising (i) a heavy chain variable (VH) region having the amino acid sequence set forth in SEQ ID NO:1, and (ii) a light chain variable (VL) region having the amino acid sequence set forth in SEQ ID NO:2, wherein the heavy and light chain framework regions are optionally modified with from 1 to 8 amino acid substitutions, deletions or insertions in the framework regions, wherein the binding agent specifically binds to human MSLN; at least one linker attached to the binding agent; and at least one cytotoxic agent attached to each linker.

In some embodiments, the binding agent comprises: (i) a heavy chain variable region having the amino acid sequence set forth in SEQ ID NO:1, and (ii) a light chain variable region having the amino acid sequence set forth in SEQ ID NO:2.

In some embodiments, a conjugate is provided comprising: a binding agent comprising a heavy chain variable (VH) region and a light chain variable (VL) region, wherein the VH region comprises a complementarity determining region HCDR1 sequence having the amino acid sequence set forth in SEQ ID NO:11, a HCDR2 having the amino acid sequence set forth in SEQ ID NO:12, and a HCDR3 having the amino acid sequence set forth in SEQ ID NO:13, each disposed within a heavy chain framework region; and wherein the VL region comprises a LCDR1 sequence having the amino acid sequence set forth in SEQ ID NO:14, a LCDR2 having the amino acid sequence set forth in SEQ ID NO:15, and a LCDR3 having the amino acid sequence set forth in SEQ ID NO:16, each disposed within a light chain framework region; at least one linker attached to the binding agent; and at least one cytotoxic agent attached to each linker.

In some embodiments, the framework regions are murine framework regions.

In some embodiments, the framework regions are human framework regions.

In some embodiments, the binding agent is an antibody or an antigen-binding portion thereof.

In some embodiments, the binding agent is a monoclonal antibody, a Fab, a Fab′, an F(ab′), an Fv, a disulfide linked Fc, a scFv, a single domain antibody, a diabody, a bi-specific antibody, or a multi-specific antibody.

In some embodiments, the heavy chain variable region further comprises a heavy chain constant region.

In some embodiments, heavy chain constant region is of the human IgG isotype.

In some embodiments, the heavy chain constant region is an IgG1 constant region.

In some embodiments, the IgG1 heavy chain constant region has the amino acid sequence set forth in positions 120-449 of SEQ ID NO:3.

In some embodiments, the heavy chain constant region is an IgG4 constant region.

In some embodiments, the heavy chain variable and constant regions have the amino acid sequence set forth in SEQ ID NO:3.

In some embodiments, the light chain variable region further comprises a light chain constant region.

In some embodiments, the light chain constant region is of the kappa isotype.

In some embodiments, the kappa light chain constant region has the amino acid sequence set forth in positions 107-213 of SEQ ID NO:4.

In some embodiments, the light chain variable and constant regions have the amino acid sequence set forth in SEQ ID NO:4.

In some embodiments, the linker is attached to the binding agent via an interchain disulfide residue, an engineered cysteine, a glycan or modified glycan, an N-terminal residue of the binding agent or a polyhistidine residue attached to the binding agent.

In some embodiments, the average drug loading of the conjugate is from about 1 to about 8, about 2, about 4, about 6, about 8, about 10, about 12, about 14, about 16, about 3 to about 5, about 6 to about 8 or about 8 to about 16.

In some embodiments, the binding agent is mono-specific.

In some embodiments, the binding agent is bivalent.

In some embodiments, the binding agent comprises a second binding domain and the binding agent is bispecific.

In some embodiments, the cytotoxic agent is selected from the group consisting of an auristatin, a camptothecin and a calicheamicin.

In some embodiments, the cytotoxic agent is an auristatin.

In some embodiments, the cytotoxic agent is monomethyl auristatin E (MMAE).

In some embodiments, the cytotoxic agent is a camptothecin.

In some embodiments, the cytotoxic agent is exatecan.

In some embodiments, the cytotoxic agent is a calicheamicin.

In some embodiments, the cytotoxic agent is SN-38 (also known as 7-Ethyl-10-hydroxycamptothecin).

In some embodiments, the linker is selected from the group consisting of me-VC-PAB, CL2, CL2A and (Succinimid-3-yl-N)—(CH2)n2-C(═O)-Gly-Gly-Phe-Gly-NH—CH2═OCH2-(C═O)—.

In some embodiments, the linker is mc-VC-PAB.

In some embodiments, the linker is attached to at least one molecule of MMAE.

In some embodiments, the linker is CL2A.

In some embodiments, the linker is attached to at least one molecule of SN-38.

In some embodiments, the linker is CL2.

In some embodiments, the linker is attached to at least one molecule of SN-38.

In some embodiments, the linker is (Succinimid-3-yl-N)—(CH2)n2-C(═O)-Gly-Gly-Phe-Gly-NH—CH2═OCH2-(C═O)—.

In some embodiments, the linker is attached to at least one molecule of exatecan.

In some embodiments, provide is a pharmaceutical composition comprising the conjugate of any of the embodiments described herein and a pharmaceutically acceptable carrier.

In some embodiments, provided is a nucleic acid encoding the binding agent of any of embodiments described herein.

In some embodiments, provided is a vector comprising the nucleic acid of the preceding embodiment.

In some embodiments, provided is a cell line comprising the nucleic acid of any of the embodiments described herein.

In some embodiments, provided is a method of treating a MSLN+ cancer, comprising administering to a subject in need thereof a therapeutically effective amount of the conjugate of any of embodiments of conjugates described herein or the pharmaceutical composition of any of these conjugates.

In some embodiments of the method, the MSLN+ cancer is a carcinoma or a malignancy.

In some embodiments of the method the MSLN+ cancer is selected from melanoma, head and neck cancer, breast cancer, mesothelioma, renal clear cell cancer, chondrosarcoma, urothelial (bladder) cancer, osteosarcoma, pancreatic cancer, and leukemia (B-ALL).

In some embodiments of the method, it further comprises administering an immunotherapy to the subject.

In some embodiments of the method, the immunotherapy comprises a checkpoint inhibitor.

In some embodiments of the method, the checkpoint inhibitor is selected from an antibody that specifically binds to human PD-1, human PD-L1, or human CTLA4.

In some embodiments of the method, the checkpoint inhibitor is pembrolizumab, nivolumab, cemiplimab or ipilimumab.

In some embodiments, the method further comprises administering chemotherapy to the subject.

In some embodiments of the method, the conjugate is administered intravenously.

In some embodiments of the method, the conjugate is administered in a dose of about 0.1 mg/kg to about 10 mg/kg or from about 0.1 mg/kg to about 12 mg/kg.

In some embodiments, provided is a method of improving treatment outcome in a subject receiving immunotherapy and/or chemotherapy for a MSLN+ cancer, comprising: administering an effective amount of an immunotherapy or chemotherapy to the subject having cancer; and administering a therapeutically effective amount of the conjugate of any of embodiments of conjugates described herein or the pharmaceutical composition of any of the conjugates described herein; wherein the treatment outcome of the subject is improved, as compared to administration of the immunotherapy or chemotherapy alone.

In some embodiments, the improved treatment outcome is an objective response selected from stable disease, a partial response or a complete response.

In some embodiments, the improved treatment outcome is reduced tumor burden.

In some embodiments, the improved treatment outcome is progression-free survival or disease-free survival.

In some embodiments, the immunotherapy is an immune checkpoint inhibitor.

In some embodiments, the immune checkpoint inhibitor comprises an antibody that specifically binds to human PD-1, human PD-L1, or CTLA4.

In some embodiments, the immune checkpoint inhibitor is pembrolizumab, nivolumab, cemiplimab or ipilimumab.

In some embodiments, the conjugate is administered intravenously.

In some embodiments, the conjugate is administered in a dose of about 0.1 mg/kg to about 10 mg/kg.

In some embodiments, provided is the use of a conjugate described herein or a pharmaceutical composition of a conjugate described herein for the treatment of MSLN+ cancer in a subject.

In some embodiments, provided is the use of a conjugate described herein or a pharmaceutical composition of any of the conjugates described herein for the treatment of MSLN+ cancer in a subject receiving immunotherapy or chemotherapy.

These and other aspects of the present disclosure may be more fully understood by reference to the following detailed description, non-limiting examples of specific embodiments and the appended drawings.

### BRIEF DESCRIPTION OF THE DRAWINGS

FIGS. 1A-1B. FACs binding of ARD110 antibody and corresponding ADCs to MSLN-positive cell lines: FIG. 1A—HCC-1806, HGC-27, NCI-N87, CaOV3, NCI-H1781, and NCI-H1975; FIG. 1B—HCC-1806 breast and NCI-H1975 lung.

FIGS. 2A-2B. Activities of ARD110-vcMMAE (FIG. 2A) and ARD110-SN38 (FIG. 2B) ADCs in an in vitro cytotoxicity assay.

FIG. 3. The antitumor effect of ARD110-vcMMAE and ARD110-SN38 ADCs in the OVCAR3 ovarian carcinoma xenograft model.

FIG. 4. The antitumor effect of ARD110-vcMMAE and ARD110-SN38 ADCs in the HCC-1806 breast carcinoma xenograft model.

FIG. 5. The antitumor effect of ARD110-vcMMAE and ARD110-SN38 ADCs in the HGC-27 gastric carcinoma xenograft model.

FIG. 6. The antitumor effect of ARD110-vcMMAE and ARD110-SN38 ADCs in the NCI-H226 mesothelioma xenograft model.

### DETAILED DESCRIPTION

The disclosure provides anti-MSLN antibodies, cytotoxic agent conjugates comprising anti-MSLN antibodies, and pharmaceutical compositions that comprise such antibodies and conjugates. The antibodies, conjugates and pharmaceutical compositions of the disclosure are useful in treating a MSLN+ cancer, alone or in combination with other cancer therapeutic agents.

For convenience, certain terms in the specification, examples and claims are defined here. Unless stated otherwise, or implicit from context, the following terms and phrases have the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein and unless otherwise indicated, the terms “a” and “an” are taken to mean “one”, “at least one” or “one or more”. Unless otherwise required by context, singular terms used herein shall include pluralities and plural terms shall include the singular.

The use of the alternative (e.g., “or”) should be understood to mean either one, both, or any combination thereof of the alternatives. As used throughout the disclosure, the terms “include” and “comprise” are used synonymously.

“Optional” or “optionally” means that the subsequently described element, component, event, or circumstance may or may not occur, and that the description includes instances in which the element, component, event, or circumstance occurs and instances in which they do not.

The phrase “at least one of” when followed by a list of items or elements refers to an open ended set of one or more of the elements in the list, which may, but does not necessarily, include more than one of the elements.

The term “about” as used throughout the disclosure in the context of a number refers to a range centered on that number and spanning 15% less than that number and 15% more than that number. The term “about” used in the context of a range refers to an extended range spanning 15% less than that the lowest number listed in the range and 15% more than the greatest number listed in the range.

Throughout the disclosure, any concentration range, percentage range, ratio range, or integer range is to be understood to include any value (including integers or fractions) or subrange within the recited range unless otherwise indicated.

Unless the context clearly requires otherwise, throughout the description and the claims, the words “comprise”. “comprising”, and the like are to be construed in an inclusive sense as opposed to an exclusive or exhaustive sense; that is to say, in the sense of “including, but not limited to”.

The terms “decrease,” “reduce,” “reduced”, “reduction”, “decrease,” and “inhibit” are all used herein generally to mean a decrease by a statistically significant amount relative to a reference.

The terms “increased”, “increase” or “enhance” or “activate” are all used herein to generally mean an increase by a statically significant amount relative to a reference.

The terms “isolated” or “partially purified” as used herein refer in the case of a nucleic acid, polypeptide or protein, to a nucleic acid, polypeptide or protein separated from at least one other component (e.g., nucleic acid or polypeptide or protein) that is present with the nucleic acid, polypeptide or protein as found in its natural source and/or that would be present with the nucleic acid, polypeptide or protein when expressed by a cell, or secreted in the case of secreted polypeptides and proteins. A chemically synthesized nucleic acid, polypeptide or protein, or one synthesized using in vitro transcription/translation, is considered “isolated.” The terms “purified” or “substantially purified” refer to an isolated nucleic acid, polypeptide or protein that is at least 95% by weight the subject nucleic acid, polypeptide or protein, including, for example, at least 96%, at least 97%, at least 98%, and at least 99% or more.

As used herein, the terms “protein” and “polypeptide” are used interchangeably herein to designate a series of amino acid residues each connected to each other by peptide bonds between the alpha-amino and carboxyl groups of adjacent residues. The terms “protein” and “polypeptide” also refer to a polymer of protein amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof.

Thus, exemplary polypeptides or proteins include gene products, naturally occurring proteins, homologs, orthologs, paralogs, fragments and other equivalents, variants, fragments, and analogs of the foregoing.

MSLN, or mesothelin, is a glycosylphosphatidylinositol-anchored cell-surface protein that may function as a cell adhesion protein. It is reported to be overexpressed on epithelial mesotheliomas, ovarian cancers and some squamous cell cancers, among other cancers.

MSLN polypeptides include, but are not limited to, those having the amino acid sequence set forth in NCBI Ref Seq. NP_005814.2 (SEQ ID NO:9) and NP_037536.2 (SEQ ID NO:10); these sequences are incorporated by reference herein.

As used herein, an “epitope” refers to the amino acids typically bound by an immunoglobulin VH/VL pair, such as the antibodies and binding agents described herein. An epitope can be formed on a polypeptide from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An epitope defines the minimum binding site for an antibody or other binding agent, and thus represent the target of specificity of an antibody, antigen binding portion thereof or other immunoglobulin-based binding agent. In the case of a single domain antibody, an epitope represents the unit of structure bound by a variable domain in isolation.

As used herein, “specifically binds” refers to the ability of a binding agent (e.g., an antibody or antigen binding portion thereof) described herein to bind to a target, such as MSLN, with a KD 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the antibody or other binding agent and the concentration of target polypeptide.

The person of ordinary skill in the art can determine appropriate conditions under which the antibodies and other binding agents described herein selectively bind to MSLN using any suitable methods, such as titration of a binding agent in a suitable cell binding assay. A binding agent specifically bound to MSLN is not displaced by a non-similar competitor. In certain embodiments, an anti-MSLN antibody or antigen-binding portion thereof is said to specifically bind to MSLN when it preferentially recognizes its target antigen, MSLN, in a complex mixture of proteins and/or macromolecules.

In some embodiments, an anti-MSLN antibody or antigen-binding portion thereof or other binding agent as described herein specifically binds to a MSLN polypeptide with a dissociation constant (KD) of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹¹ M. or less. In some embodiments, an anti-MSLN antibody or antigen-binding portion thereof or other binding agent as described herein specifically binds to a MSLN polypeptide with a dissociation constant (KD) of from about 10⁻⁵ M to 10⁻⁶ M. In some embodiments, an anti-MSLN antibody or antigen-binding portion thereof or other binding agent as described herein specifically binds to a MSLN polypeptide with a dissociation constant (KD) of from about 10⁻⁶ M to 10⁻⁷ M. In some embodiments, an anti-MSLN antibody or antigen-binding portion thereof or other binding agent as described herein specifically binds to a MSLN polypeptide with a dissociation constant (KD) of from about 10⁻⁷ M to 10⁻⁸ M. In some embodiments, an anti-MSLN antibody or antigen-binding portion thereof or other binding agent as described herein specifically binds to a MSLN polypeptide with a dissociation constant (KD) of from about 10⁻⁸ M to 10⁻⁹ M. In some embodiments, an anti-MSLN antibody or antigen-binding portion thereof or other binding agent as described herein specifically binds to a MSLN polypeptide with a dissociation constant (KD) of from about 10⁻⁹ M to 10⁻¹⁰ M. In some embodiments, an anti-MSLN antibody or antigen-binding portion thereof or other binding agent as described herein specifically binds to a MSLN polypeptide with a dissociation constant (KD) of from about 10⁻¹⁰ M to 10⁻¹¹ M. In some embodiments, an anti-MSLN antibody or antigen-binding portion thereof or other binding agent as described herein specifically binds to a MSLN polypeptide with a dissociation constant (KD) of from about 10⁻¹¹ M to 10⁻¹² M. In some embodiments, an anti-MSLN antibody or antigen-binding portion thereof or other binding agent as described herein specifically binds to a MSLN polypeptide with a dissociation constant (KD) of less than 10⁻¹² M.

As used throughout the disclosure, “identical” or “identity” refer to the similarity between a DNA, RNA, nucleotide, amino acid, or protein sequence to another DNA, RNA, nucleotide, amino acid, or protein sequence. Identity can be expressed in terms of a percentage of sequence identity of a first sequence to a second sequence. Percent (%) sequence identity with respect to a reference DNA sequence can be the percentage of DNA nucleotides in a candidate sequence that are identical with the DNA nucleotides in the reference DNA sequence after aligning the sequences. Percent (%) sequence identity with respect to a reference amino acid sequence can be the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity. As used throughout the disclosure, the percent sequence identity values is generated using the NCBI BLAST 2.0 software as defined by Altschul et al., “Gapped BLAST and PSI-BLAST: a new generation of protein database search programs,” Nucleic Acids Res. 2007, 25, 3389-3402, with the parameters set to default values.

As used herein, the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

Other than in the examples, or where otherwise indicated, all numbers expressing quantities of ingredients or reaction conditions used herein should be understood as modified in all instances by the term “about.” The term “about” when used in connection with percentages can mean+/−1%.

The term “statistically significant” or “significantly” refers to statistical significance and generally means a two standard deviation (2SD) difference, above or below a reference value.

Other terms are defined herein within the description of the various aspects of the disclosure.

### I. Antibodies

Provided herein are ARD110 binding antibodies (also referred to as anti-MSLN antibodies or MSLN binding antibodies) and antigen binding portions thereof that specifically bind to mesothelin (MSLN). Also provided herein are conjugates of ARD110 (MSLN binding) antibodies and antigen binding portions and cytotoxic agents (also referred to as MSLN conjugates). In some embodiments, the MSLN conjugates reduce the number of MSLN+ cancer cells in a subject.

In some embodiments, the MSLN antibody or antigen binding portion thereof comprises (i) a heavy chain variable region having the amino acid sequence set forth in SEQ ID NO:1, and (ii) a light chain variable region having the amino acid sequence set forth in SEQ ID NO:2. In some embodiments, the MSLN binding antibody or antigen binding portion thereof comprises (i) a heavy chain variable region having the amino acid sequence set forth in SEQ ID NO:1 and (ii) a light chain variable region having the amino acid sequence set forth in SEQ ID NO:2, wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the MSLN antibody or antigen binding portion thereof comprises (i) a heavy chain variable region having the amino acid sequence set forth in SEQ ID NO:1 and (ii) a light chain variable region having the amino acid sequence set forth in SEQ ID NO:2, wherein the heavy and light chain variable framework regions are optionally modified with from 1 to 8, 1 to 6, 1 to 4 or 1 to 2 amino acid substitutions, deletions or insertions in the framework regions, and wherein the CDRs of the heavy or light chain variable regions are not modified.

In some embodiments, provided herein is a binding agent comprising (i) a heavy chain variable region having the amino acid sequence set forth in SEQ ID NO:1, and (ii) a light chain variable region having the amino acid sequence set forth in SEQ ID NO:2, wherein the binding agent specifically binds to MSLN In some embodiments, provided herein is a binding agent comprising (i) a heavy chain variable region having the amino acid sequence set forth in SEQ ID NO:1 and (ii) a light chain variable region having the amino acid sequence set forth in SEQ ID NO:2, wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising (i) a heavy chain variable region having the amino acid sequence set forth in SEQ ID NO:1 and (ii) a light chain variable region having the amino acid sequence set forth in SEQ ID NO:2, and wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified. As described herein, a binding agent includes an anti-MSLN antibody or antigen binding portion(s) thereof and can include other peptides or polypeptides covalently attached to the MSLN antibody or antigen binding portion thereof. In any of these embodiments, the binding agent specifically binds to MSLN

In some embodiments, the heavy and/or light chain CDRs of an antibody or antigen binding fragment thereof may be identified by using any one of the following methods: Kabat, Chothia, AbM, Contact, IMGT, and/or Aho.

In some embodiments, provided is a binding agent comprising a heavy chain variable (VH) region and a light chain variable (VL) region, wherein the VH region comprises a complementarity determining region heavy chain complementarity determining region 1 (HCDR1) having the amino acid sequence set forth in SEQ ID NO:11, a heavy chain complementarity determining region 2 (HCDR2) having the amino acid sequence set forth in SEQ ID NO:12 and a heavy chain complementarity determining region 3 (HCDR3) having the amino acid sequence set forth in SEQ ID NO:13, and the VL region comprises a light chain complementarity determining region 1 (LCDR1) having the amino acid sequence set forth in SEQ ID NO:14, a light chain complementarity determining region 2 (LCDR2) having the amino acid sequence set forth in SEQ ID NO:15, and a light chain complementarity determining region 3 (LCDR3) having the amino acid sequence set forth in SEQ ID NO:16, and wherein each VH and VL comprises a humanized framework region and the binding agent specifically binds to MSLN.

In some embodiments, the compositions and methods described herein relate to reduction of MSLN+ cells in a subject (e.g., reducing the number of MSLN+ cells in a cancer or tumor) by an anti-MSLN antibody, antigen binding portion thereof, other binding agent or conjugate thereof in vivo. In some embodiments, the compositions and methods described herein relate to the treatment of MSLN+ cancer in a subject by administering an anti-MSLN antibody, antigen binding portion thereof, other binding agent or conjugate thereof.

As used herein, the term “antibody” refers to immunoglobulin molecules and immunologically active portions of immunoglobulin molecules, i.e., molecules that contain an antigen binding site that specifically binds to an antigen. The term generally refers to antibodies comprised of two immunoglobulin heavy chain variable regions and two immunoglobulin light chain variable regions including full length antibodies (having heavy and light chain constant regions) and antigen-binding portions thereof; including, for example, an intact monoclonal antibody, a Fab, a Fab′, a F(ab′), a Fv, a disulfide linked Fv, a scFv, a single domain antibody (dAb), a diabody, a multi-specific antibody, a dual specific antibody, a bispecific antibody, and single chain antibodies (see, e.g., Huston et al., Proc. Natl. Acad. Sci. USA., 85, 5879-5883 (1988) and Bird et al., Science 242, 423-426 (1988), which are incorporated herein by reference). An antibody can include, for example, polyclonal, monoclonal, and genetically engineered antibodies, and antigen binding fragments thereof. An antibody can be, for example, murine, chimeric, humanized, heteroconjugate, bispecific, diabody, triabody, or tetrabody.

Each heavy chain is typically composed of a variable region (abbreviated as VH) and a constant region. The heavy chain constant region may include three domains CH1, CH2 and CH3 and optionally a fourth domain, CH4. Each light chain is typically composed of a variable region (abbreviated as VL) and a constant region. The light chain constant region is a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs that are arranged from the N terminus to the C terminus in the following order: FR1, CDR1, FR2, CDR2, FR3, CDR3, and FR4. This structure is well known to those skilled in the art. CDR and FR sequences may be determined by several different numbering schemes, including Kabat, Chothia, AbM, Contact, IMGT, and/or Aho.

In some embodiments, an antigen binding portion comprises a light chain complementary determining region 1 (LCDR1), a light chain complementary determining region 2 (LCDR2), a light chain complementary determining region 3 (LCDR3), a heavy chain complementary determining region 1 (HCDR1), a heavy chain complementary determining region 2 (HCDR2), and a heavy chain complementary determining region 3 (HCDR3).

The amino acid sequences of the VH CDRs of the MSLN antibody are set forth in SEQ ID NO:1 at amino acids 31-35 (GYTMN, HCDR1, SEQ ID NO:11), 50-66 (LITPYNGASSYNQKFRG, HCDR2, SEQ ID NO:12) and 99-108 (GGYDGRGFDY, HCDR3, SEQ ID NO:13). The amino acid sequences of the VL CDRs of the MSLN antibody are set forth in SEQ ID NO:2 at amino acids 24-33 (SASSSVSYMH, LCDR1, SEQ ID NO:14), 49-55 (DTSKLAS, LCDR2, SEQ ID NO:15) and 88-96 (QQWSKHPLT, LCDR3, SEQ ID NO:16) The phrase “wherein the CDRs of the heavy or light chain variable regions are not modified” refers to these VH and VL CDRs (SEQ ID NOs:11-16), which do not have amino acid substitutions, deletions or insertions.

As used herein, an “antigen-binding portion” or “antigen-binding fragment” of an anti-MSLN antibody refers to a region of an antibody molecule that specifically binds to an antigen. In some embodiments, the antigen-binding portion refers to the portions of an anti-MSLN antibody as described herein having the VH and VL sequences of the MSLN antibody (set forth in SEQ ID NO:1 and SEQ ID NO:2, optionally modified as described herein). In accordance with the term “antigen-binding portion” of an antibody, examples of antigen binding portions include a Fab, a Fab′, a F(ab′)², a Fv, a disulfide linked Fv, a scFv, a single domain antibody (dAb), a diabody, heavy chain antibody (hcAb), VHH, VNAR, nanobody, and single chain antibodies. As used herein, the terms Fab, F(ab′)2 and Fv refer to the following. (i) an Fab fragment, i.e. a monovalent fragment composed of the VL, VH, CL and CH1 domains; (ii) an F(ab′)2 fragment, i.e. a bivalent fragment comprising two Fab fragments linked to one another in the hinge region via a disulfide bridge: and (iii) an Fv fragment composed of the VL and VH domains of an anti-MSLN antibody. Although the two domains of the Fv fragment, namely VL and VH, are encoded by separate coding regions, they may further be linked to one another using a synthetic linker, e.g. a poly-G4S amino acid sequence (‘(G4S)n’ disclosed as SEQ ID NO:17, wherein n=1 to 5), making it possible to prepare them as a single protein chain in which the VL and VH regions combine in order to form monovalent molecules (known as single chain Fv (ScFv)). The term “antigen-binding portion” of an antibody is also intended to include such single chain antibodies. Other forms of single chain antibodies such as “diabodies” are likewise included here Diabodies are bivalent, bispecific antibodies in which VH and VL domains are expressed on a single polypeptide chain, but using a linker connecting the VH and VL domains that is too short for the two domains to be able to combine on the same chain, thereby forcing the VH and VL domains to pair with complementary domains of a different chain (VL and VH, respectively), and to form two antigen-binding sites (see, for example, Holliger, R, et al. (1993) Proc. Natl. Acad. Sci. USA 90:64446448; Poljak, R. J, et al. (1994) Structure 2:1121-1123).

An immunoglobulin constant region refers to a heavy or light chain constant region. The constant region provide the general framework of the antibody and may not be involved directly in binding the antibody to an antigen, but can be involved in various effector functions, such as participation of the antibody in antibody-dependent cellular cytotoxicity (ADCC), ADCP (antibody-dependent cellular phagocytosis), CDC (complement-dependent cytotoxicity) and complement fixation, binding to Fc receptors (e.g., CD16, CD32, FcRn), greater in vivo half-life relative to a polypeptide lacking an Fc region, protein A binding, and perhaps even placental transfer (see Capon et al., Nature 337:525, 1989). As used throughout the disclosure, “Fc region” refers to the heavy chain constant region segment of the Fc fragment (the “fragment crystallizable” region or Fc region) from an antibody, which can in include one or more constant domains, such as CH2, CH3, CH4, or any combination thereof. In some embodiments, an Fc region includes the CH2 and CH3 domains of an IgG. IgA, or IgD antibody, or the CH3 and CH4 domains of an IgM or IgE antibody.

Human heavy chain and light chain constant region amino acid sequences are known in the art A constant region can be of any suitable type, which can be selected from the classes of immunoglobulins, IgA, IgD, IgE, IgG, and IgM. Several immunoglobulin classes can be further divided into isotypes, e.g., IgG1, IgG2, IgG3, IgG4, or IgA1, and IgA2 The heavy-chain constant regions (Fc) that corresponds to the different classes of immunoglobulins can be α, δ, ε, γ, and μ, respectively. The light chains can be one of either kappa (or κ) and lambda (or λ).

In some embodiments, a constant region can have an IgG1 isotype. In some embodiments, a constant region can have an IgG2 isotype. In some embodiments, a constant region can have an IgG3 isotype. In some embodiments, a constant region can have an IgG4 isotype. In some embodiments, an Fc region can have a hybrid isotype comprising constant domains from two or more isotypes. In some embodiments, an immunoglobulin constant region can be an IgG1 or IgG4 constant region.

In some embodiments, an anti-MSLN antibody has an IgG1 heavy chain constant region. In some embodiments, an IgG1 heavy chain constant region has the amino acid sequence set forth in positions 120-449 of SEQ ID NO:3. In some embodiments, an anti-MSL antibody has a kappa light chain constant region. In some embodiments, a kappa light chain constant region has the amino acid sequence set forth in positions 107-213 of SEQ ID NO:4.

In some embodiments, an anti-MSLN antibody heavy chain is of the IgG1 isotype and has the amino acid sequence set forth in SEQ ID NO:7. In some embodiments, an anti-MSLN antibody light chain is of the kappa isotype and has the amino acid sequence set forth in SEQ ID NO:8.

Furthermore, an anti-MSLN antibody or an antigen-binding portion thereof may be part of a larger binding agent formed by covalent or noncovalent association of the antibody or antibody portion with one or more other proteins or peptides. Relevant to such binding agents are the use of the streptavidin core region in order to prepare a tetrameric scFv molecule (Kipriyanov, S. M., et al. (1995), Human Antibodies and Hybridomas 6:93-101) and the use of a cysteine residue, a marker peptide and a C-terminal polyhistidinyl peptide, e.g. hexahistidinyl tag (‘hexahistidinyl tag’ disclosed as SEQ ID NO:18) in order to produce bivalent and biotinylated scFv molecules (Kipriyanov, S. M., et al. (1994) Mol. Immunol. 31:10471058).

As to the VH and VL amino acid sequences, one of skill in the art will recognize that individual substitutions, deletions or additions (insertions) to a nucleic acid encoding the VH or VL, or amino acids in polypeptide that alter a single amino acid or a small percentage of amino acids in the encoded sequence is a “conservatively modified variant”, where the alteration results in the substitution of an amino acid with a chemically similar amino acid (a conservative amino acid substitution) and the altered polypeptide retains the ability to specifically bind to MSLN.

In some embodiments, a conservatively modified variant of an anti-MSLN antibody or antigen binding portion thereof can have alterations in the framework regions (FR); i.e., other than in the CDRs), e.g. a conservatively modified variant of an anti-MSLN antibody has the amino acid sequences of the VH and VL CDRs (set forth in SEQ ID NOs:11-16) and has at least one conservative amino acid substitution in the FR. In some embodiments, the VH and VL amino acid sequences (set forth in SEQ ID NOs: 1 and 2, respectively) collectively have no more than 8 or 6 or 4 or 2 or 1 conservative amino acid substitutions in the FR, as compared to the amino acid sequences of the VH and VL (SEQ ID NOs: 1 and 2, respectively). In some embodiments, the VH and VL amino acid sequences (set forth in SEQ ID NOs: 1 and 2, respectively) have 8 to 1, 6 to 1, 4 to 1 or 2 to 1 conservative amino acid substitutions in the FR, as compared to the amino acid sequences of the VH and VL (set forth in SEQ ID NOs: 1 and 2, respectively). In further aspects of any of these embodiments, a conservatively modified variant of the anti-MSLN antibody, antigen binding portion thereof or other binding agent exhibits specific binding to MSLN.

For conservative amino acid substitutions, a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative amino acid substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained, i.e., to MSLN.

For conservative substitutions, amino acids can be grouped according to similarities in the properties of their side chains (in A L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M), (2) uncharged polar: Gly (G), Ser (S), Thr (T), Cys (C), Tyr (Y), Asn (N), Gln (Q); (3) acidic: Asp (D). Glu (E); and (4) basic: Lys (K), Arg (R), His (H).

Alternatively, for conservative substitutions naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic Asp, Glu; (4) basic: His, Lys, Arg; (5) residues that influence chain orientation: Gly, Pro; and (6) aromatic: Trp, Tyr, Phe. Non-conservative substitutions will entail exchanging a member of one of these classes or another class.

Particular conservative substitutions include, for example: Ala to Gly or to Ser; Arg to Lys; Asn to Gln or to His; Asp to Glu; Cys to Ser; Gln to Asn; Glu to Asp; Gly to Ala or to Pro; His to Asn or to Gln; Ile to Leu or to Val; Leu to Ile or to Val; Lys to Arg; to Gln or to Glu; Met to Leu; to Tyr or to Ile; Phe to Met; to Leu or to Tyr; Ser to Thr; Thr to Ser; Tip to Tyr; Tyr to Trp; and/or Phe to Val; to Ile or to Leu.

In some embodiments, a conservatively modified variant of an anti-MSLN antibody or antigen binding portion thereof preferably is at least 90%, at least 91%, at least 92%, at least 93%, at least 94%, at least 95%, at least 96%, at least 97%, at least 98%, at least 99%, or more, identical to the reference VH or VL sequence, wherein the VH and VL CDRs (SEQ ID NOs:11-16) are not modified. As used throughout the disclosure, “identical” or “identity” refer to the similarity between a DNA, RNA, nucleotide, amino acid, or protein sequence to another DNA, RNA, nucleotide, amino acid, or protein sequence. Identity can be expressed in terms of a percentage of sequence identity of a first sequence to a second sequence. Percent (%) sequence identity with respect to a reference DNA sequence can be the percentage of DNA nucleotides in a candidate sequence that are identical with the DNA nucleotides in the reference DNA sequence after aligning the sequences. Percent (%) sequence identity with respect to a reference amino acid sequence can be the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity. As used throughout the disclosure, the percent sequence identity values is generated using the NCBI BLAST 2.0 software as defined by Altschul et al., “Gapped BLAST and PSI-BLAST: a new generation of protein database search programs,” Nucleic Acids Res. 2007, 25, 3389-3402, with the parameters set to default values.

In some embodiments, the VH and VL amino acid sequences (set forth in SEQ ID NOs:1 and 2, respectively) collectively have no more than 8 or 6 or 4 or 2 or 1 conservative amino acid substitutions in the framework regions, as compared to the amino acid sequences of the VH and VL (set forth in SEQ ID NOs: 1 and 2, respectively). In some embodiments, the VH and VL amino acid sequences (set forth in SEQ ID Nos: 1 and 2, respectively) collectively have 8 to 1, or 6 to 1, or 4 to 1, or 2 to 1 conservative amino acid substitutions in the framework regions, as compared to the amino acid sequences of the VH and VL (set forth in SEQ ID NOs: 1 and 2, respectively). In some embodiments, the VH and VL amino acid sequences (set forth in SEQ ID NOs: 1 and 2, respectively) collectively have no more than 8 or 6 or 4 or 2 or 1 amino acid substitutions, deletions or insertions in the framework regions, as compared to the amino acid sequences of the VH and VL (set forth in SEQ ID NOs: 1 and 2, respectively). In some embodiments, the VH and VL amino acid sequences (set forth in SEQ ID NOs: 1 and 2, respectively) have 8 to 1, 6 to 1, 4 to 1, or 2 to 1 conservative amino acid substitutions in the framework regions, as compared to the amino acid sequences of the VH and VL (set forth in SEQ ID NOs: 1 and 2, respectively). In some embodiments, the VH and VL amino acid sequences (set forth in SEQ ID NOs:1 and 2, respectively) collectively have no more than 8 or 6 or 4 or 2 or 1 amino acid substitutions, deletions or insertions, as compared to the amino acid sequences of the VH and VL (set forth in SEQ ID NOs:1 and 2, respectively).

Modification of a native (or reference) amino acid sequence can be accomplished by any of a number of techniques known to one of skill in the art Mutations can be introduced, for example, at particular loci by synthesizing oligonucleotides containing the desired mutant sequence, flanked by restriction sites enabling ligation to fragments of the native sequence. Following ligation, the resulting reconstructed sequence encodes a variant having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion desired. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al (Genetic Engineering Principles and Methods, Plenum Press, 1981); and U.S. Pat. Nos. 4,518,584 and 4,737,462, which are herein incorporated by reference in their entireties.

In some embodiments, an anti-MSLN antibody or antigen-binding portion thereof has fully human constant regions. In some embodiments, an anti-MSLN antibody or antigen-binding portion thereof has non-human constant regions. In some embodiments, an anti-MSLN antibody heavy chain is of the IgG1 isotype and has the amino acid sequence set forth in SEQ ID NO:7. In some embodiments, an anti-MSLN antibody light chain is of the kappa isotype and has the amino acid sequence set forth in SEQ ID NO:8.

In various embodiments, anti-MSLN antibodies, antigen binding portions thereof and other binding agents can be produced in human, murine or other animal-derived cells lines.

Recombinant DNA expression can be used to produce anti-MSLN antibodies, antigen binding portions thereof and other binding agents. This allows the production of anti-MSLN antibodies as well as a spectrum of anti-MSLN antigen binding portions and other binding agents (including fusion proteins) in a host species of choice. The production of anti-MSLN antibodies, antigen binding portions thereof and other binding agents in bacteria, yeast, transgenic animals and chicken eggs are also alternatives for cell-based production systems. The main advantages of transgenic animals are potential high yields from renewable sources.

In some embodiments, an anti-MSLN VH polypeptide having the amino acid sequence set forth in SEQ ID NO:1 is encoded by a nucleic acid. In some embodiments, an anti-MSLN VL polypeptide having the amino acid sequence set forth in SEQ ID NO:2 is encoded by a nucleic acid. In some embodiments, an anti-MSLN VH polypeptide having the amino acid sequence set forth in SEQ ID NO:1 is encoded by a nucleic acid having the sequence set forth in SEQ ID NO:21. In some embodiments, an anti-MSLN VL polypeptide having the amino acid sequence set forth in SEQ ID NO. 2 is encoded by a nucleic acid having the sequence set forth in SEQ ID NO:22.

As used herein, the term “nucleic acid” or “nucleic acid sequence” or “polynucleotide sequence” or “nucleotide” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA If single stranded, a nucleic acid may be the coding strand or non-coding (anti-sense strand). A nucleic acid molecule may contain natural subunits or non-natural subunits. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing. In some embodiments, the nucleic acid can be a cDNA, e.g., a nucleic acid lacking introns.

Nucleic acid molecules encoding the amino acid sequence of an anti-MSLN antibody, antigen binding portion thereof as well as other binding agents can be prepared by a variety of methods known in the art. These methods include, but are not limited to, preparation of synthetic nucleotide sequences encoding of an anti-MSLN antibody, antigen binding portion or other binding agent(s). In addition, oligonucleotide-mediated (or site-directed) mutagenesis, PCR-mediated mutagenesis, and cassette mutagenesis can be used to prepare nucleotide sequences encoding an anti-MSLN antibody or antigen binding portion as well as other binding agents. A nucleic acid sequence encoding at least an anti-MSLN antibody, antigen binding portion thereof, binding agent, or a polypeptide thereof, as described herein, can be recombined with vector DNA in accordance with conventional techniques, such as, for example, blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, e.g., by Maniatis et al., Molecular Cloning, Lab. Manual (Cold Spring Harbor Lab. Press, N Y, 1982 and 1989), and Ausubel et al., Current Protocols in Molecular Biology (John Wiley & Sons), 1987-1993, and can be used to construct nucleic acid sequences and vectors that encode an anti-MSLN antibody or antigen binding portion thereof or a VH or VL polypeptide thereof.

A nucleic acid molecule, such as DNA, is said to be “capable of expressing” a polypeptide if it contains nucleotide sequences that contain transcriptional and translational regulatory information and such sequences are “operably linked” to nucleotide sequences that encode the polypeptide. An operable linkage is a linkage in which the regulatory DNA sequences and the DNA sequence sought to be expressed (e.g., an anti-MSLN antibody or antigen binding portion thereof) are connected in such a way as to permit gene expression of a polypeptide(s) or antigen binding portions in recoverable amounts. The precise nature of the regulatory regions needed for gene expression may vary from organism to organism, as is well known in the analogous art. See, e.g., Sambrook et al., 1989; Ausubel et al., 1987-1993.

Accordingly, the expression of an anti-MSLN antibody or antigen-binding portion thereof as described herein can occur in either prokaryotic or eukaryotic cells. Suitable hosts include bacterial or eukaryotic hosts, including yeast, insects, fungi, bird and mammalian cells either in vivo or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of an anti-MSLN antibody or antigen binding portion thereof as described herein with a specified amino terminus sequence. Moreover, problems associated with retention of initiation codon-derived methionine residues in direct yeast (or bacterial) expression maybe avoided. (See, e.g., Sabin et al., 7 Bio/Technol. 705 (1989). Miller et al., 7 Bio/Technol. 698 (1989).) Any of a series of yeast gene expression systems incorporating promoter and termination elements from the actively expressed genes coding for glycolytic enzymes produced in large quantities when yeast are grown in medium rich in glucose can be utilized to obtain recombinant anti-MSLN antibodies or antigen-binding portions thereof. Known glycolytic genes can also provide very efficient transcriptional control signals. For example, the promoter and terminator signals of the phosphoglycerate kinase gene can be utilized.

Production of anti-MSLN antibodies or antigen-binding portions thereof in insects can be achieved, for example, by infecting an insect host with a baculovirus engineered to express a polypeptide by methods known to those of ordinary skill in the art. See Ausubel et al., 1987-1993.

In some embodiments, the introduced nucleic acid sequence (encoding an anti-MSLN antibody or antigen binding portion thereof or a polypeptide thereof) is incorporated into a plasmid or viral vector capable of autonomous replication in a recipient host cell. Any of a wide variety of vectors can be employed for this purpose and are known and available to those of ordinary skill in the art. See, e.g., Ausubel et al., 1987-1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Exemplary viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox) Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B N. Fields el al., Eds., Lippincott-Raven Publishers, Philadelphia, 1996). In some such embodiments, the viral vector is a lentiviral vector or a γ-retroviral vector.

Exemplary prokaryotic vectors known in the art include plasmids such as those capable of replication in E. coli. Other gene expression elements useful for the expression of DNA encoding anti-MSLN antibodies or antigen-binding portions thereof include, but are not limited to (a) viral transcription promoters and their enhancer elements, such as the SV40 early promoter. (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983). Immunoglobulin-encoding DNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin encoding nucleotide sequences, the transcriptional promoter can be, for example, human cytomegalovirus, the promoter enhancers can be cytomegalovirus and mouse/human immunoglobulin.

In some embodiments, for expression of DNA coding regions in rodent cells, the transcriptional promoter can be a viral LTR sequence, the transcriptional promoter enhancers can be either or both the mouse immunoglobulin heavy chain enhancer and the viral LTR enhancer, and the polyadenylation and transcription termination regions. In other embodiments, DNA sequences encoding other proteins are combined with the above-recited expression elements to achieve expression of the proteins in mammalian cells.

Each coding region or gene fusion is assembled in, or inserted into, an expression vector. Recipient cells capable of expressing the anti-MSLN variable region(s) or antigen binding portions thereof (e.g., a VH having the amino acid sequence set forth in SEQ ID NO:1 and/or a VL having the amino acid sequence set forth in SEQ ID NO:2 or a variant thereof as described herein) are then transfected singly with nucleotides encoding an anti-MSLN antibody or an antibody polypeptide or antigen-binding portion thereof, or are co-transfected with a polynucleotide(s) encoding VH and a VL chain coding regions. The transfected recipient cells are cultured under conditions that permit expression of the incorporated coding regions and the expressed antibody chains or intact antibodies or antigen binding portions are recovered from the culture.

In some embodiments, the nucleic acids containing the coding regions encoding an anti-MSLN antibody or antigen-binding portion thereof (e.g., a VH having the amino acid sequence set forth in SEQ ID NO:1 and/or a VL having the amino acid sequence set forth in SEQ ID NO:2 or a variant thereof as described herein) are assembled in separate expression vectors that are then used to co-transfect a recipient host cell. Each vector can contain one or more selectable genes. For example, in some embodiments, two selectable genes are used, a first selectable gene designed for selection in a bacterial system and a second selectable gene designed for selection in a eukaryotic system, wherein each vector has a set of coding regions. This strategy results in vectors which first direct the production, and permit amplification, of the nucleotide sequences in a bacterial system. The DNA vectors so produced and amplified in a bacterial host are subsequently used to co-transfect a eukaryotic cell, and allow selection of a co-transfected cell carrying the desired transfected nucleic acids (e.g., containing anti-MSLN antibody heavy and light chains) Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively the fused nucleotide sequences encoding VH and VL chains can be assembled on the same expression vector.

For transfection of the expression vectors and production of the anti-MSLN antibodies or antigen binding portions thereof, the recipient cell line can be a Chinese Hamster ovary cell line (e.g., DG44) or a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0. SP2/0 cells only produce immunoglobulins encoded by the transfected genes. Myeloma cells can be grown in culture or in the peritoneal cavity of a mouse, where secreted immunoglobulin can be obtained from ascites fluid.

An expression vector encoding an anti-MSLN antibody or antigen-binding portion thereof (e.g., a VH having the amino acid sequence set forth in SEQ ID NO:1 and/or a VL having the amino acid sequence set forth in SEQ ID NO:2 or a variant thereof as described herein) can be introduced into an appropriate host cell by any of a variety of suitable means, including such biochemical means as transformation, transfection, protoplast fusion, calcium phosphate-precipitation, and application with polycations such as diethylaminoethyl (DEAE) dextran, and such mechanical means as electroporation, direct microinjection and microprojectile bombardment. Johnston et al., 240 Science 1538 (1988), as known to one of ordinary skill in the art.

Yeast provides certain advantages over bacteria for the production of immunoglobulin heavy and light chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes polypeptides bearing leader sequences (i.e., pre-polypeptides). See, e.g., Hitzman et al., 11th Intl. Conf. Yeast, Genetics & Molec. Biol. (Montpelier, France, 1982).

Yeast gene expression systems can be routinely evaluated for the levels of production, secretion and the stability of antibodies, and assembled anti-MSLN antibodies and antigen binding portions thereof. Various yeast gene expression systems incorporating promoter and termination elements from the actively expressed genes coding for glycolytic enzymes produced in large quantities when yeasts are grown in media rich in glucose can be utilized.

Known glycolytic genes can also provide very efficient transcription control signals. For example, the promoter and terminator signals of the phosphoglycerate kinase (PGK) gene can be utilized. Another example is the translational elongation factor 1 alpha promoter A number of approaches can be taken for evaluating optimal expression plasmids for the expression of immunoglobulins in yeast. See II DNA Cloning 45, (Glover, ed., IRL Press, 1985) and e.g., U.S. Publication No. US 2006/0270045 A1.

Bacterial strains can also be utilized as hosts for the production of the antibody molecules or antigen binding portions thereof described herein, E. coli K12 strains such as E. coli W3110, Bacillus species, enterobacteria such as Salmonella typhimurium or Serratia marcescens, and various Pseudomonas species can be used Plasmid vectors containing replicon and control sequences which are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of anti-MSLN antibodies and antigen binding portions thereof in bacteria (see Glover, 1985; Ausubel, 1987, 1993, Sambrook, 1989; Colligan, 1992-1996).

Host mammalian cells can be grown in vitro or in vivo. Mammalian cells provide post-translational modifications to immunoglobulin molecules including leader peptide removal, folding and assembly of VH and VL chains, glycosylation of the antibody molecules, and secretion of functional antibody and/or antigen binding portions thereof.

Mammalian cells which can be useful as hosts for the production of antibody proteins, in addition to the cells of lymphoid origin described above, include cells of fibroblast origin, such as Vero or CHO-K1 cells. Exemplary eukaryotic cells that can be used to express immunoglobulin polypeptides include, but are not limited to, COS cells, including COS 7 cells: 293 cells, including 293-6E cells; CHO cells, including CHO-S and DG44 cells; PERC6™ cells (Crucell); and NSO cells. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

In some embodiments, one or more anti-MSLN antibodies or antigen-binding portions thereof (e.g., a VH having the amino acid sequence set forth in SEQ ID NO:1 and/or a VL having the amino acid sequence set forth in SEQ ID NO:2 or a variant thereof as described herein) can be produced in vivo in an animal that has been engineered or transfected with one or more nucleic acid molecules encoding the polypeptides, according to any suitable method.

In some embodiments, an antibody or antigen-binding portion thereof (e.g., a VH having the amino acid sequence set forth in SEQ ID NO:1 and/or a VL having the amino acid sequence set forth in SEQ ID NO:2 or a variant thereof as described herein) is produced in a cell-free system. Non-limiting exemplary cell-free systems are described, e.g., in Sitaraman et al., Methods Mol. Biol. 498: 229-44 (2009); Spirin, Trends Biotechnol 22: 538-45 (2004), Endo et al., Biotechnol. Adv. 21: 695-713 (2003).

Many vector systems are available for the expression of the VH and VL chains (e.g., a VH having the amino acid sequence set forth in SEQ ID NO:1 and/or a VL having the amino acid sequence set forth in SEQ ID NO:2 or a variant thereof as described herein) in mammalian cells (see Glover, 1985). Various approaches can be followed to obtain intact antibodies. As discussed above, it is possible to co-express VH and VL chains and optionally the associated constant regions in the same cells to achieve intracellular association and linkage of VH and VL chains into complete tetrameric H₂L₂ antibodies or antigen-binding portions thereof. The co-expression can occur by using either the same or different plasmids in the same host. Nucleic acids encoding the VH and VL chains or antigen binding portions thereof (e.g., a VH having the amino acid sequence set forth in SEQ ID NO:1 and a VL having the amino acid sequence set forth in SEQ ID NO:2 or a variant thereof as described herein) can be placed into the same plasmid, which is then transfected into cells, thereby selecting directly for cells that express both chains. Alternatively, cells can be transfected first with a plasmid encoding one chain, for example the VL chain, followed by transfection of the resulting cell line with a VH chain plasmid containing a second selectable marker. Cell lines producing antibodies, antigen-binding portions thereof via either route could be transfected with plasmids encoding additional copies of peptides, VH, VL, or VH plus VL chains (e.g., a VH having the amino acid sequence set forth in SEQ ID NO:1 and/or a VL having the amino acid sequence set forth in SEQ ID NO:2 or a variant thereof as described herein) in conjunction with additional selectable markers to generate cell lines with enhanced properties, such as higher production of assembled anti-MSLN antibodies or antigen binding portions thereof or enhanced stability of the transfected cell lines.

Additionally, plants have emerged as a convenient, safe and economical alternative expression system for recombinant antibody production, which are based on large scale culture of microbes or animal cells. Anti-MSLN antibodies or antigen binding portions can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560, 6,512,162; WO 0129242. Several plant-derived antibodies have reached advanced stages of development, including clinical trials (see, e.g., Biolex, N.C.).

For intact antibodies, the variable regions (VH and VL) of the anti-MSLN antibodies (e.g., a VH having the amino acid sequence set forth in SEQ ID NO:1 and/or a VL having the amino acid sequence set forth in SEQ ID NO:2 or a variant thereof as described herein) are typically linked to at least a portion of an immunoglobulin constant region (e.g., Fc), typically that of a human immunoglobulin. Human constant region DNA sequences can be isolated in accordance with well-known procedures from a variety of human cells, such as immortalized B-cells (WO 87/02671; which is incorporated by reference herein in its entirety). An anti-MSLN binding antibody can contain both light chain and heavy chain constant regions. The heavy chain constant region can include CH1, hinge, CH2, CH3, and, sometimes, CH4 regions. In some embodiments, the CH2 domain can be deleted or omitted.

Alternatively, techniques described for the production of single chain antibodies (see, e.g. U.S. Pat. No. 4,946,778; Bird, Science 242: 423-42 (1988), Huston et al., Proc. Natl. Acad. Sci. USA 85:5879-5883 (1988); and Ward et al., Nature 334:544-54 (1989); which are incorporated by reference herein in their entireties) can be adapted to produce single chain antibodies that specifically bind to MSLN. Single chain antibodies are formed by linking the heavy and light chain variable regions (e.g., having the amino acid sequences set forth in SEQ ID NO:1 and 2, or a variant thereof as described herein (e.g., optionally modified with from 1 to 8 amino acid substitutions, deletions and/or insertions)) of the Fv region via an amino acid bridge, resulting in a single chain polypeptide. Techniques for the assembly of functional Fv fragments in E. coli can also be used (see, e.g. Skerra et al., Science 242:1038-1041 (1988), which is incorporated by reference herein in its entirety).

Intact (e.g., whole) antibodies, their dimers, individual light and heavy chains, or antigen binding portions thereof can be recovered and purified by known techniques, e.g., immunoadsorption or immunoaffinity chromatography, chromatographic methods such as HPLC (high performance liquid chromatography), ammonium sulfate precipitation, gel electrophoresis, or any combination of these. See generally, Scopes, Protein Purification (Springer-Verlag, N.Y., 1982). Substantially pure MSLN binding antibodies or antigen binding portions thereof of at least about 90% to 95% homogeneity are advantageous, as are those with 98% to 99% or more homogeneity, particularly for pharmaceutical uses. Once purified, partially or to homogeneity as desired, an intact anti-MSLN antibody or antigen binding portions thereof can then be used therapeutically or in developing and performing assay procedures, immunofluorescent staining, and the like. See generally, Vols. I & II Immunol. Meth. (Lefkovits & Pernis, eds., Acad. Press, N Y, 1979 and 1981).

Additionally, and as described herein, an anti-MSLN antibody or antigen binding portion thereof can be further optimized to decrease potential immunogenicity, while maintaining functional activity, for therapy in humans. In some embodiments, an optimized MSLN binding antibody or antigen binding portion thereof is derived from an anti-MSLN antibody comprising (i) a heavy chain variable region having the amino acid sequence set forth in SEQ ID NO:1 and (ii) a light chain variable region having the amino acid sequence set forth in SEQ ID NO:2, wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, an optimized MSLN binding antibody or antigen binding portion thereof is derived from a MSLN binding antibody comprising i) a heavy chain variable region having the amino acid sequence set forth in SEQ ID NO:1 and (ii) a light chain variable region having the amino acid sequence set forth in SEQ ID NO:2, wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. In this regard, functional activity means an anti-MSLN antibody or antigen binding portion thereof capable of displaying one or more known functional activities associated with a MSLN binding antibody or antigen binding portion thereof comprising (i) a heavy chain variable region having the amino acid sequence set forth in SEQ ID NO:1 and (ii) a light chain variable region having the amino acid sequence set forth in SEQ ID NO:2 In any of these embodiments, the functional activity of the MSLN binding antibody or antigen binding portion thereof includes specifically binding to MSLN Additional functional activities include anti-cancer activity. Additionally, an anti-MSLN antibody or antigen binding portion thereof having functional activity means the polypeptide exhibits activity similar to, or better than, the activity of a reference antibody or antigen-binding portion thereof as described herein (e.g., a MSLN binding antibody or antigen binding portion thereof comprising (i) a heavy chain variable region having the amino acid sequence set forth in SEQ ID NO:1 and (ii) a light chain variable region having the amino acid sequence set forth in SEQ ID NO:2 or a variant thereof, as described herein), as measured in a particular assay, such as, for example, a biological assay, with or without dose dependency. In the case where dose dependency does exist, it need not be identical to that of the reference antibody or antigen-binding portion thereof, but rather substantially similar to or better than the dose-dependence in a given activity as compared to the reference antibody or antigen-binding portion thereof as described herein (i.e., the candidate polypeptide will exhibit greater activity relative to the reference antibody).

### II. Antibody Drug Conjugates

In some embodiments, the anti-MSLN (ARD110) antibody is part of an anti-MSLN antibody drug conjugate (or MSLN conjugate). In some embodiments, the anti-MSLN antibody is attached to at least one linker, and at least one cytotoxic agent is attached to each linker.

As used herein, a “cytotoxic agent refers to a compound that exerts a cytotoxic or cytostatic effect on a cell, e.g., by preventing cell growth or replication A “small molecule” or “compound” is an organic compound with a molecular weight of less than 1500, or 100, or 900, or 750, or 600, or 500 Daltons. A “small molecule drug” is a small molecule that has a therapeutic effect such as treating a disease or disorder. In some embodiments, a small molecule is not a protein, a polysaccharide, or a nucleic acid.

In some embodiments, a cytotoxic agent is microtubule disrupting agent (e.g., tubulin disrupting agent) or a DNA modifying agent.

In some embodiments, the MSLN conjugate includes a cytotoxic agent that is a tubulin disrupting agent. Several different categories of tubulin disrupting agent are known, including, auristatins, tubulysins, colchicine, vinca alkaloids, taxanes, cryptophycins, maytansinoids, hemiasterlins, as well as other tubulin disrupting agents. Auristatins are derivatives of the natural product dolastatin 10. Exemplary auristatins include MMAE (N-methylvaline-valine-dolaisoleuine-dolaproine-norephedrine or monomethyl auristatin E) and MMAF (N-methylvaline-valine-dolaisoleuine-dolaproine-phenylalanine or monomethyl auristatin F) and AFP (see WO2004/010957 and WO2007/008603). WO 2015/057699 describes PEGylated auristatins including MMAE. Additional dolastatin derivatives contemplated for use are disclosed in U.S. Pat. No. 9,345,785, incorporated herein by reference.

Tubulysins include, but are not limited to, tubulysin D, tubulysin M, tubuphenylalanine and tubutyrosine WO 2017-096311 and WO 2016-040684 describe tubulysin analogs including tubulysin M.

Colchicines include, but are not limited to, colchicine and CA-4.

Vinca alkaloids include, but are not limited to, vinblastine (VBL), vinorelbine (VRL), vincristine (VCR) and vindesine (VOS).

Taxanes include, but are not limited to, paclitaxel and docetaxel.

Cryptophycins include but are not limited to cryptophycin-1 and cryptophycin-52 Maytansinoids include, but are not limited to, maytansine, maytansinol, maytansine analogs in DM1, DM3 and DM4, and ansamatocin-2. Exemplary maytansinoid drug moieties include those having a modified aromatic ring, such as: C-19-dechloro (U.S. Pat. No. 4,256,746) (prepared by lithium aluminum hydride reduction of ansamitocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides), and those having modifications at other positions.

Maytansinoid drug moieties also include those having modifications such as: C-9-SH (U.S. Pat. No. 4,424,219) (prepared by the reaction of maytansinol with H₂S or P₂S₅); C-14-alkoxymethyl(demethoxy/CH2OR) (U.S. Pat. No. 4,331,598); C-14-hydroxymethyl or acyloxymethyl (CH₂OH or CH₂OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudiflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAI reduction of maytansinol) The cytotoxicity of the TA.1-maytansonoid conjugate that binds HER-2 (Chai et al., Cancer Research 52:127-131 (1992) was tested in vitro on the human breast cancer cell line SK-BR-3. The drug conjugate achieved a degree of cytotoxicity similar to the free maytansinoid drug, which could be increased by increasing the number of maytansinoid molecules per antibody molecule.

Hemiasterlins include but are not limited to, hemiasterlin and HTI-286.

Other tubulin disrupting agents include taccalonolide A, taccalonolide B, taccalonolide AF, taccalonolide AJ, taccalonolide Al-epoxide, discodermolide, epothilone A, epothilone B, and laulimalide.

In some embodiments, the cytotoxic agent is a DNA modifying agent. In some embodiments, the DNA modifying agent is an alkylating agent or topoisomerase inhibitor. In some embodiments, a DNA modifying agent is a duocarmycin analog, calicheamicin, or pyrrolobenzodiazepine,

In some embodiments, the cytotoxic agent can be a topoisomerase inhibitor, such as a camptothecin, such as camptothecin, irinotecan (also referred to as CPT-11), topotecan, 10-hydroxy-CPT, SN-38, exatecan and the exatecan analog DXd (see US20150297748).

The MSLN conjugates contemplated for use in the methods herein comprise at least one linker, each linker having at least one cytotoxic agent attached to it. Typically, the conjugate includes a linker between the anti-MSLN antibody or antigen binding fragment thereof and the cytotoxic agent. The linker may be a protease cleavable linker (see, e.g., WO2004/010957), an acid-cleavable linker, a disulfide linker, self-stabilizing linker (see, e.g., WO2018/031690 and WO2015/095755), a non-cleavable linker (see, e.g., WO2007/008603), and/or a hydrophilic linker (see, e.g., WO2015/123679). In various embodiments, the linker is cleavable under intracellular conditions, such that cleavage of the linker releases the cytotoxic agent from the antibody in the intracellular environment.

For example, in some embodiments, the linker is cleavable by a cleaving agent that is present in the intracellular environment (e.g., within a lysosome or endosome or caveolea) The linker can be, e.g., a peptidyl linker that is cleaved by an intracellular peptidase or protease enzyme, including, but not limited to, a lysosomal or endosomal protease. Typically, a peptidyl linker is at least one amino acid long or at least two amino acids long Cleaving agents can include cathepsins B and D and plasmin, all of which are known to hydrolyze dipeptide drug derivatives resulting in the release of active drug inside target cells (see, e.g., Dubowchik and Walker, 1999, Pharm Therapeutics 83.67-123). Most typical are peptidyl linkers that are cleavable by enzymes that are present in target antigen-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker) Other such linkers are described, e.g., in U.S. Pat. No. 6,214,345. In specific embodiments, the peptidyl linker cleavable by an intracellular protease is a Val-Cit linker or a Phe-Lys linker (see, e.g., U.S. Pat. No. 6,214,345, which describes the synthesis of doxoubicin with the val-cit linker) or Gly-Gly-Phe-Gly linker (see, e.g., US Patent Publication 2015/0297748). One advantage of using intracellular proteolytic release of the cytotoxic agent is that the agent is typically attenuated when conjugated and the serum stabilities of the conjugates are typically high. See also U.S. Pat. No. 9,345,785.

As used herein, the terms “intracellularly cleaved” and “intracellular cleavage” refer to a metabolic process or reaction inside a cell on an antibody drug conjugate, whereby the covalent attachment, e.g., the linker, between the cytotoxic agent and the antibody is broken, resulting in the free cytotoxic agent, or other metabolite of the conjugate dissociated from the antibody inside the cell. The cleaved moieties of the conjugate are thus intracellular metabolites.

In some embodiments, the cleavable linker is pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; and 5,622,929; Dubowchik and Walker, 1999, Pharm Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929)).

In various embodiments, the linker is cleavable under reducing conditions (e.g., a disulfide linker) A variety of disulfide linkers are known, including, for example, those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT (see, e.g., Thorpe et al., 1987, Cancer Res. 47:5924-5931; Wawrzynczak et al., In Immunoconjugates. Antibody Conjugates in Radioimagery and Therapy of Cancer (C W Vogel ed., Oxford U. Press, 1987. See also U.S. Pat. No. 4,880,935.)

In various embodiments, the linker is a malonate linker (Johnson et al., 1995, Anticancer Res. 15:1387-93), a maleimidobenzoyl linker (Lau et al., 1995. Bioorg-Med-Chem. 3(10):1299-1304), or a 3′-N-amide analog (Lau et al., 1995, Bioorg-Med-Chem. 3(10):1305-12). In some embodiments, the linker unit is not cleavable and the drug is released by antibody degradation. (See U.S. Publication No. 2005/0238649).

In various embodiments, a linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of the antibody drug conjugate (ADC) or ADC derivative, are cleaved when the ADC or ADC derivative is present in an extracellular environment (e.g., in plasma) Whether a linker is not substantially sensitive to the extracellular environment can be determined, for example, by incubating independently with plasma both (a) the ADC or ADC derivative (the “ADC sample”) and (b) an equal molar amount of unconjugated antibody or therapeutic agent (the “control sample”) for a predetermined time period (e.g., 2, 4, 8, 16, or 24 hours) and then comparing the amount of unconjugated antibody or therapeutic agent present in the ADC sample with that present in control sample, as measured, for example, by high performance liquid chromatography.

In various embodiments, the linker promotes cellular internalization. In certain embodiments, the linker promotes cellular internalization when conjugated to the cytotoxic agent (i.e., in the milieu of the linker-therapeutic agent moiety of the ADC or ADC derivative as described herein). In yet other embodiments, the linker promotes cellular internalization when conjugated to both the cytotoxic agent and the anti-MSLN antibody or derivative thereof (i.e., in the milieu of the ADC or ADC derivative as described herein).

A variety of linkers that can be used with the present compositions and methods are described in PCT Publication WO 2004/010957. In various embodiments, the protease cleavable linker comprises a thiol-reactive spacer and a dipeptide. In some embodiments, the protease cleavable linker consists of a thiol-reactive maleimidocaproyl spacer, a valine-citrulline dipeptide, and a p-amino-benzyloxycarbonyl spacer.

In various embodiments, the acid cleavable linker is a hydrazine linker or a quaternary ammonium linker (see PCT Publication WO2017/096311 and WO2016/040684.)

Self-stabilizing linkers comprising a maleimide group are described in U.S. Pat. No. 9,504,756.

In various embodiments, a tubulin disrupting agent, such as an auristatin, is conjugated to a linker by a C-terminal carboxyl group that forms an amide bond with the Linker Unit (LU) as described in U.S. Pat. No. 9,463,252, incorporated herein by reference. In various embodiments, the Linker unit comprises at least one amino acid Binder-drug conjugates (ADCs) of N,N-dialkylauristatins are disclosed in U.S. Pat. No. 8,992,932

In various embodiments, the linker also comprises a stretcher unit and/or an amino acid unit Exemplary stretcher units and amino acid units are described in U.S. Pat. Nos. 9,345,785 and 9,078,931, each of which is herein incorporated by reference.

In various embodiments, provided herein is the use of antibody drug conjugates comprising an anti-MSLN antibody, covalently linked to MMAE through an mc-val-cit-PAB linker. The MSLN conjugates are delivered to the subject as a pharmaceutical composition.

In some embodiments, the MSLN conjugates have the following formula:

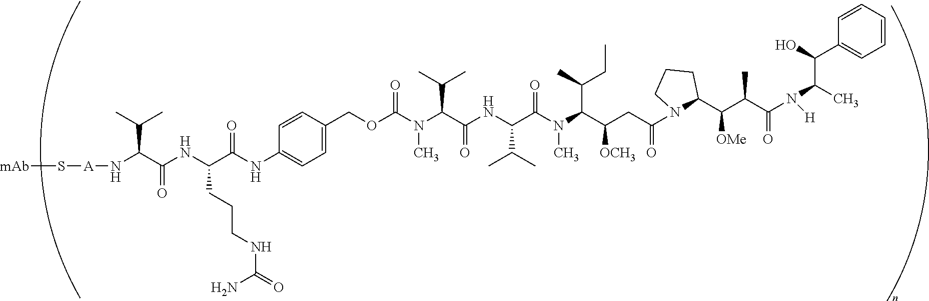

or a pharmaceutically acceptable salt thereof wherein: mAb is an anti-MSLN antibody, S is a sulfur atom of the antibody, A− is a Stretcher unit, and n is from about 3 to about 5, or from about 3 to about 8.

The drug loading is represented by p, the average number of drug molecules (cytotoxic agents) per antibody in a pharmaceutical composition. For example, if p is about 4, the average drug loading taking into account all of the antibody present in the pharmaceutical composition is about 4. In some embodiments, P ranges from about 3 to about 5, more preferably from about 3.6 to about 4.4, even more preferably from about 3.8 to about 4.2. P can be about 3, about 4, or about 5. In some embodiments, P ranges from about 6 to about 8, more preferably from about 7.5 to about 8.4. P can be about 6, about 7, or about 8. The average number of drugs per antibody in preparation of conjugation reactions may be characterized by conventional means such as mass spectroscopy, ELISA assay, and HPLC The quantitative distribution of antibody-drug conjugates in terms of p may also be determined. In some instances, separation, purification, and characterization of homogeneous antibody-drug-conjugates where p is a certain value from antibody-drug-conjugates with other drug loadings may be achieved by means such as reverse phase HPLC or electrophoresis.

A Stretcher unit (A) is capable of linking an antibody unit to an amino acid unit (e.g., a valine-citrulline peptide) via a sulfhydryl group of the antibody Sulfhydryl groups can be generated, for example, by reduction of the interchain disulfide bonds of an anti-MSLN antibody. For example, a Stretcher unit can be linked to the antibody via the sulfur atoms generated from reduction of the interchain disulfide bonds of the antibody. In some embodiments, the Stretcher units are linked to the antibody solely via the sulfur atoms generated from reduction of the interchain disulfide bonds of the antibody. In some embodiments, sulfhydryl groups can be generated by reaction of an amino group of a lysine moiety of an anti-MSLN antibody with 2-iminothiolane (Traut's reagent) or other sulfhydryl generating reagents. In certain embodiments, the anti-MSLN antibody is a recombinant antibody and is engineered to carry one or more lysines. In certain other embodiments, the recombinant anti-MSLN antibody is engineered to carry additional sulfhydryl groups, e.g., additional cysteines.

The synthesis and structure of MMAE is described in U.S. Pat. No. 6,884,869 incorporated by reference herein in its entirety and for all purposes. The synthesis and structure of exemplary Stretcher units and methods for making antibody drug conjugates are described in, for example, U.S. Publication Nos. 2006/0074008 and 2009/0010945 each of which is incorporated herein by reference in its entirety.

Representative Stretcher units are described within the square brackets of Formulas IIIa and IIIb of U.S. Pat. No. 9,211,319, and incorporated herein by reference.

In various embodiments, the antibody drug conjugate comprises monomethyl auristatin E and a protease-cleavable linker. It is contemplated that the protease cleavable linker comprises a thiol-reactive spacer and a dipeptide. In various embodiments, the protease cleavable linker consists of a thiol-reactive maleimidocaproyl spacer, a valine-citrulline dipeptide, and a p-amino-benzyloxycarbonyl or PAB spacer.

The abbreviation “MMAE” refers to monomethyl auristatin E.

The abbreviations “vc” and “vat-cit” refer to the dipeptide valine-citrulline.

The abbreviation “PAB” refers to the self-immolative spacer:

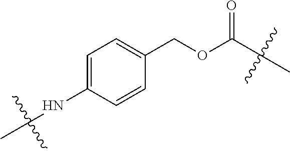 [SMILES: *Nc1ccc(COC(*)=O)cc1]

The abbreviation “MC” refers to the stretcher maleimidocaproyl:

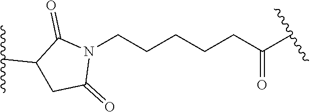 [SMILES: *C(=O)CCCCCN1C(=O)CC(*)C1=O]

In other exemplary embodiments, the conjugate has the following general formula:

Ab-[L3]-[L2]-[L]ₘ-AAₙ-cytotoxic agent,

where Ab is an anti-MSLN antibody: the cytotoxic agent can be a tubulin-disrupting agent or topoisomerase inhibitor; L3 is a component of a linker comprising an antibody-coupling moiety and one or more of acetylene (or azide) groups; L2 comprises a defined PEG (polyethylene glycol) azide (or acetylene) at one end, complementary to the acetylene (or azide) moiety in L3, and a reactive group such as carboxylic acid or hydroxyl group at the other end; L1 comprises a collapsible unit (e.g., a self-immolative group(s)), or a peptidase-cleavable moiety optionally attached to a collapsible unit, or an acid-cleavable moiety; AA is an amino acid; m is an integer with values of 0 or 1, and n is an integer with values of 0, 1, 2, 3, or 4 Such linkers can be assembled via click chemistry (See, e.g., U.S. Pat. Nos. 7,591,944 and 7,999,083)

In some embodiments, the cytotoxic agent is a camptothecin or a camptothecin (CPT) analog, such as irinotecan (also referred to as CPT-11), topotecan, 10-hydroxy-CPT, exatecan, DXd and SN-38 Representative structures are shown below.

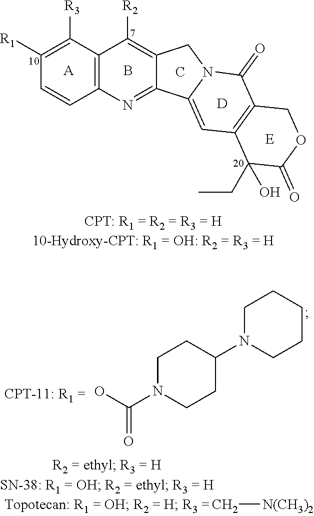

Referring to the conjugate formula Ab-[L3]-[L2]-[L1]ₘ-AAₙ-cytotoxic agent, in some embodiments, m is 0. In such embodiments, an ester moiety is first formed between the carboxylic acid of an amino acid (AA) such as glycine, alanine, or sarcosine, or of a peptide such as glycylglycine, and a hydroxyl group of a cytotoxic agent. In this example, the N-terminus of the amino acid or polypeptide may be protected as a Boc or a Fmoc or a monomethoxytrityl (MMT) derivative, which is deprotected after formation of an ester bond with the hydroxyl group of the cytotoxic agent. Selective removal of amine-protecting group, in the presence of a BOC protecting group at a hydroxyl position of the cytotoxic agent containing an additional hydroxyl group(s) can be achieved using monomethoxytrityl (MMT) as the protecting group for the amino group of amino acid or polypeptide involved in ester formation, since ‘MMT’ is removable by mild acid treatment such as dichloroacetic acid that does not cleave a BOC group. After the amino group of the amino acid or polypeptide, forming an ester bond with hydroxyl of the cytotoxic agent, is demasked, the amino group is reacted with the activated form of a COOH group on PEG moiety of L2 under standard amide-forming conditions. In a preferred embodiment, L3 comprises a thiol-reactive group which links to thiol groups of the antibody. The thiol-reactive group is optionally a maleimide or vinylsulfone, or bromoacetamide, or iodoacetamide, which links to a thiol group of the antibody. In some embodiments, the reagent bearing a thiol-reactive group is generated from succinimidyl-4-(N maleimidomethyl)cyclohexane-1-carboxylate (SMCC) or from succinimidyl-(epsilon-maleimido)caproate, for instance, with the thiol-reactive group being a maleimide group.

In another embodiments, m is 0, and AA comprises a peptide moiety, preferably a di, tri or tetrapeptide, that is cleavable by intracellular peptidase such as Cathepsin-B. Examples of cathepsin-B-cleavable peptides are: Phe-Lys, Val-Cit (Dubowchick, 2002), Ala-Leu, Leu-Ala-Leu, and Ala-Leu-Ala-Leu (Trouet et al., 1982).

In a preferred embodiment, L1 is composed of intracellularly-cleavable peptide, such as cathepsin-B-cleavable peptide, connected to the collapsible unit p-aminobenzyl alcohol (or p-amino-benzyloxycarbonyl) at the peptide's C-terminus, the benzyl alcohol portion of which is in turn directly attached to a hydroxyl group of the cytotoxic agent, in chloroformate form. In this embodiment, n is 0. Alternatively, when n is non-zero, the benzyl alcohol portion of the p-amidobenzyl alcohol (or p-amino-benzyloxycarbonyl) moiety is attached to the N-terminus of the amino acid or peptide linking at the hydroxyl group of the cytotoxic agent through the activated form of p-amidobenzyl alcohol, namely PABOCOPNP where PNP is p-nitrophenyl. In a preferred embodiment, the linker comprises a thiol-reactive group which links to thiol groups of the antibody. The thiol-reactive group is optionally a maleimide or vinylsulfone, or bromoacetamide, or iodoacetamide, which links to thiol groups of the antibody. In a preferred embodiment, the component bearing a thiol-reactive group is generated from succinimidyl-4-(N maleimidomethyl)cyclohexane-1-carboxylate (SMCC) or from succinimidyl-(epsilon-maleimido)caproate, for instance, with the thiol-reactive group being a maleimide group.

In a preferred embodiment, where the cytotoxic agent is a camptothecin or analog or derivative thereof having a 20-hydroxyl, L is composed of intracellularly-cleavable peptide, such as cathepsin-B-cleavable peptide, connected to the collapsible linker p-aminobenzyl alcohol (or p-amino-benzyloxycarbonyl) at the peptide's C-terminus, the benzyl alcohol portion of which is in turn directly attached to CPT-20-O-chloroformate. In this embodiment, n is 0. Alternatively, when ‘n’ is non-zero, the benzyl alcohol portion of the p-amidobenzyl alcohol moiety is attached to the N-terminus of the amino acid or polypeptide linking at CPT's 20 position through the activated form of p-amidobenzyl alcohol, namely PABOCOPNP where PNP is p-nitrophenyl. In a preferred embodiment, the linker comprises a thiol-reactive group which links to thiol groups of an antibody. The thiol-reactive group is optionally a maleimide or vinylsulfone, or bromoacetamide, or iodoacetamide, which links to thiol groups of an antibody. In a preferred embodiment, the component bearing a thiol-reactive group is generated from succinimidyl-4-(N maleimidomethyl)cyclohexane-1-carboxylate (SMCC) or from succinimidyl-(epsilon-maleimido)caproate, for instance, with the thiol-reactive group being a maleimide group.

In another embodiment, the L2 component of the conjugate contains a polyethylene glycol (PEG) spacer that can be of up to MW 5000 in size, and in a preferred embodiment, PEG is a defined PEG with (1-12 or 1-30) repeating monomeric units. In a further preferred embodiment, PEG is a defined PEG with 1-12 repeating monomeric units. The introduction of PEG may involve using heterobifunctionalized PEG derivatives which are available commercially. In the context of the present disclosure, the heterobifunctional PEG contains an azide or acetylene group. An example of a heterobifunctional defined PEG containing 8 repeating monomeric units, with ‘NHS’ being succinimidyl, is given below in the following formula:

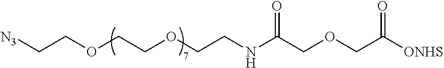

In a preferred embodiment, L3 has a plurality of acetylene (or azide) groups, ranging from 2-40, but preferably 2-20, and more preferably 2-5, and a single antibody binding moiety.

A representative conjugate, in which the cytotoxic agent is SN-38 (a CPT analog), prepared with a maleimide-containing SN-38-linker derivative, with the bonding to an antibody (designated MAb) represented as a succinimide, is given below Here, m=0, and the 20-O-AA ester bonding to SN-38 is glycinate; azide-acetylene coupling joining of L2 and L3 results in the triazole moiety as shown.

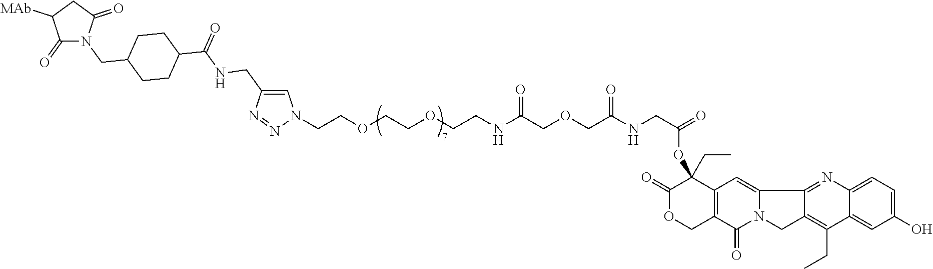

In another representative conjugate, prepared with a maleimide-containing SN-38-linker derivative, with the bonding to an antibody (MAb) represented as a succinimide, is shown below. Here, n=0 in the general formula 2; ‘L1’ contains a cathepsin-B-cleavable dipeptide attached to the collapsible p-aminobenzyl alcohol moiety, and the latter is attached to SN-38 as a carbonate bonding at the 20 position; azide-acetylene coupling joining the ‘L2’ and ‘L3’ parts results in the triazole moiety as shown.

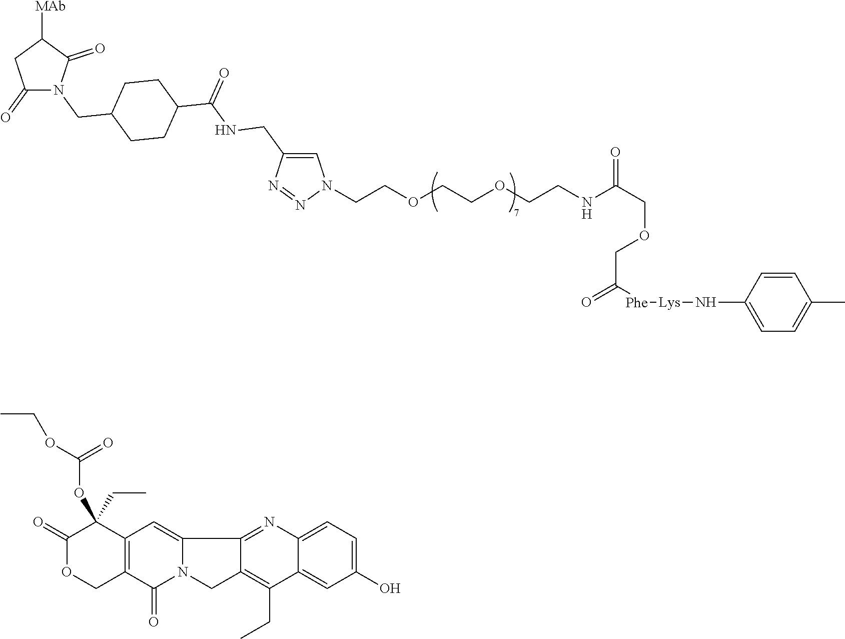

Another representative SN-38 conjugate, Mab-CL2-SN-38, prepared with a maleimide-containing SN-38-linker derivative, with the bonding to an antibody represented as a succinimide, is given below. Here, the 20-O-AA ester bonding to SN-38 is glycinate that is attached to L1 portion via a p-aminobenzyl alcohol moiety and a cathepsin-B-cleavable dipeptide; the latter is in turn attached to ‘L2’ via an amide bond, while ‘L2’ and ‘L3’ parts are coupled via azide-acetylene ‘click chemistry’.

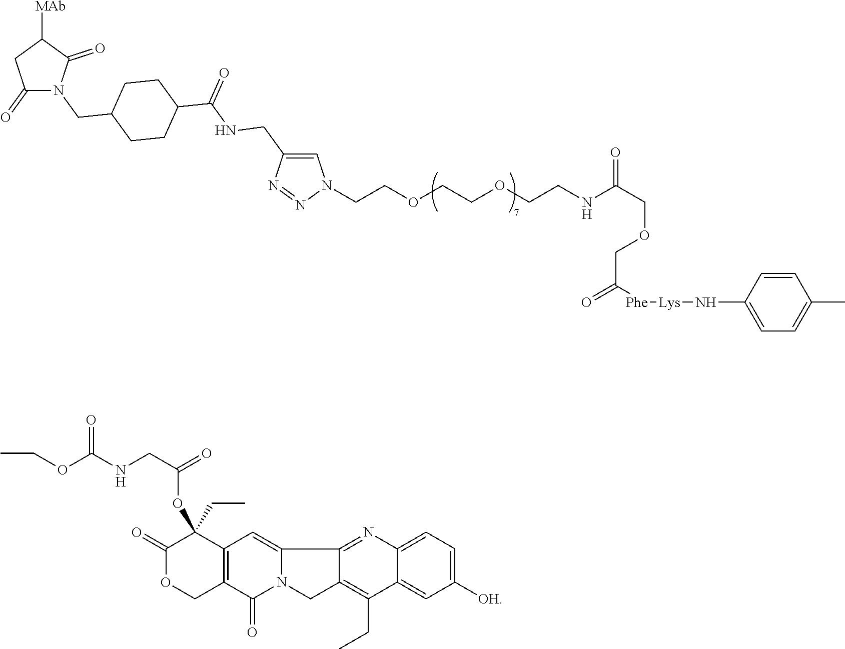

In another example of a preferred embodiment is given below. ‘L1’ contains a single amino acid attached to the collapsible p-aminobenzyl alcohol moiety, where the p-aminobenzyl alcohol is substituted or unsubstituted (R), where m=1 and n=0 in the general conjugate formula, and the cytotoxic agent is exemplified with SN-38 The structure is represented below (referred to as MA b-CL X-SN-38). Single amino acid of AA can be selected from any one of the following L-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. The substituent R on 4-aminobenzyl alcohol moiety is hydrogen or an alkyl group selected from C1-C10 alkyl groups.

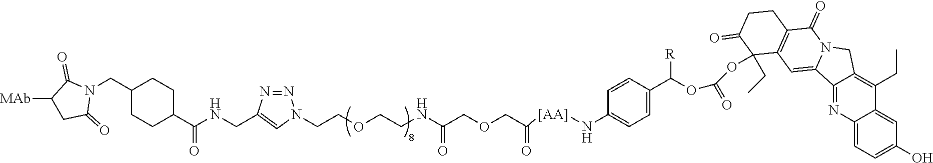

An embodiment of MAb-CLX-SN-38 (above), wherein the single amino acid AA is L-lysine and R═H, and the cytotoxic agent is exemplified by SN-38 (referred to as MAb-CL2A-SN-38) is shown below:

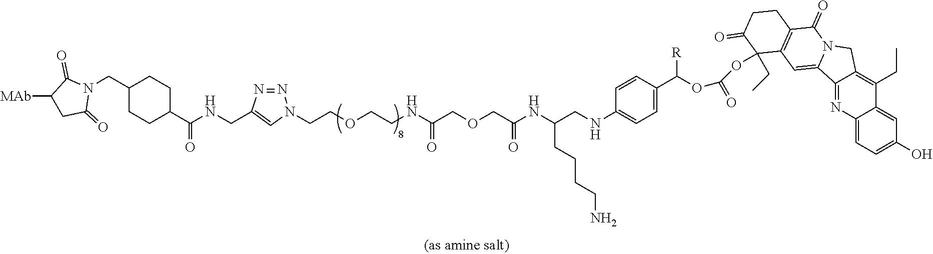

In other embodiments, a cytotoxic agent is attached to a linker comprising a Stretcher unit (Z) attached to an Amino Acid unit (AA) attached to a Spacer unit (Y), where the Stretcher unit is attached to the antibody (Ab or MAb) and the Spacer unit is attached to an amino group of a cytotoxic agent. Such a linker has the following formula:

Ab-Z-AA-Y-cytotoxic agent,

where Z is selected from -(Succinimid-3-yl-N)—(CH₂)ₙ²—C(═O)—, —CH₂—C(═O)—NH—(CH₂)n³-C(═O)—, —C(═O)-cyc.Hex(1,4)-CH₂—(N-ly-3-diminiccuS)-, or —C(═O)—(CH₂)n⁴-C(═O)—, wherein n² represents an integer of 2 to 8, n³ represents an integer of 1 to 8, and n⁴ represents an integer of 1 to 8; cyc.Hex(1,4) represents a 1,4-cyclohexylene group, and (N-ly-3-diminiccuS)- has a structure represented by the following formula:

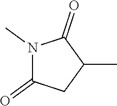

AA is a peptide of from 2 to 7 amino acids. The spacer unit Y is —NH—(CH₂)_{b}—(C═O)— or —NH—CH₂—O—CH₂—(C═O)—, where b is an integer from 1 to 5.

In some embodiments, the cytotoxic agent is exatecan. In some embodiments, the amino acid unit (AA) is -Gly-Gly-Phe-Gly-. In some embodiments, the spacer unit Y is —NH—CH₂—O—CH₂—(C═O)—.

In some embodiments, the linker-cytotoxic agent has the following structure:

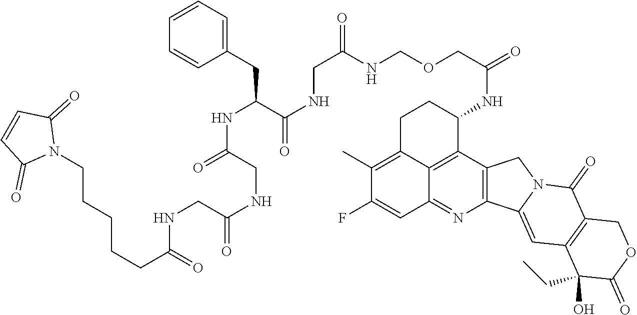 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

where the released cytotoxic agent is DXd (see U.S. Pat. No. 9,808,537).

### III. Attachment of Cytotoxic Agent-Linkers to Antibodies or Antibody Binding Portions

Techniques for attaching cytotoxic agents to antibodies or antigen binding portions thereof via linkers are well-known in the art. See, e.g., Alley et al., Current Opinion in Chemical Biology 2010 14:1-9; Senter, Cancer J., 2008, 14(3):154-169. In some embodiments, a linker is first attached to a cytotoxic agent(s) and then the linker-cytotoxic agent(s) is attached to the antibody or antigen binding portion thereof. In some embodiments, a linker is first attached to an antibody or antigen binding portion thereof, and then a cytotoxic agent(s) is attached to the linker. In the following discussion, the term linker-cytotoxic agent(s) is used to exemplify attachment of linkers or linker-cytotoxic agent(s) to antibodies or antigen binding portions thereof; the skilled artisan will appreciate that the selected attachment method can be selected according to linker and the cytotoxic agent. In some embodiments, a cytotoxic agent is attached to an antibody or antigen binding portion thereof via a linker in a manner that reduces its activity until it is released from the conjugate (e.g., by hydrolysis, by proteolytic degradation or by a cleaving agent).

Generally, a conjugate may be prepared by several routes employing organic chemistry reactions, conditions, and reagents known to those skilled in the art, including: (1) reaction of a nucleophilic group of an antibody or antigen binding portion thereof with a bivalent linker reagent to form an antibody-linker intermediate via a covalent bond, followed by reaction with a cytotoxic agent, and (2) reaction of a nucleophilic group of a cytotoxic agent with a bivalent linker reagent, to form linker-cytotoxic agent(s), via a covalent bond, followed by reaction with a nucleophilic group of an antibody or antigen binding portion thereof. Exemplary methods for preparing conjugates via the latter route are described in U.S. Pat. No. 7,498,298, which is expressly incorporated herein by reference.

Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides, (ii) alkyl and benzyl halides such as haloacetamides; and (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent such as DTT (dithiothreitol) or tricarbonylethylphosphine (TCEP), such that the antibody is fully or partially reduced. Each cysteine bridge will thus form, theoretically, two reactive thiol nucleophiles. Additional nucleophilic groups can be introduced into antibodies through modification of lysine residues, e.g., by reacting lysine residues with 2-iminothiolane (Traut's reagent), resulting in conversion of an amine into a thiol. Reactive thiol groups may also be introduced into an antibody by introducing one, two, three, four, or more cysteine residues (e.g., by preparing variant antibodies comprising one or more non-native cysteine amino acid residues).

Conjugates of the disclosure may also be produced by reaction between an electrophilic group on an antibody, such as an aldehyde or ketone carbonyl group, with a nucleophilic group on a linker reagent or drug. Useful nucleophilic groups on a linker reagent include, but are not limited to, hydrazide, oxime, amino, hydrazine, thiosemicarbazone, hydrazine carboxylate, and arylhydrazide. In one embodiment, an antibody is modified to introduce electrophilic moieties that are capable of reacting with nucleophilic substituents on the linker reagent or drug. In another embodiment, the sugars of glycosylated antibodies may be oxidized. e.g. with periodate oxidizing reagents, to form aldehyde or ketone groups which may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody or antigen binding portion thereof that can react with appropriate groups on the drug (see, e.g., Hermanson, Bioconjugate Techniques). In another embodiment, antibodies containing N-terminal serine or threonine residues can react with sodium meta-periodate, resulting in production of an aldehyde in place of the first amino acid (Geoghegan & Stroh, (1992) Bioconjugate Chem. 3:138-146, U.S. Pat. No. 5,362,852). Such an aldehyde can be reacted with a cytotoxic agent or linker.

Exemplary nucleophilic groups on a cytotoxic agent include, but are not limited to: amine, thiol, hydroxyl, hydrazide, oxime, hydrazine, thiosemicarbazone, hydrazine carboxylate, and arythydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Nonlimiting exemplary cross-linker reagents that may be used to prepare a conjugate are described herein or are known to persons of ordinary skill in the art. Methods of using such cross-linker reagents to link two moieties, including a proteinaceous moiety and a chemical moiety, are known in the art. In some embodiments, a fusion protein comprising an antibody and a cytotoxic agent may be made, e.g., by recombinant techniques or peptide synthesis. A recombinant DNA molecule may comprise regions encoding the antibody and cytotoxic portions of the conjugate either adjacent to one another or separated by a region encoding a linker peptide which does not destroy the desired properties of the conjugate.

In yet another embodiment, an antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a drug or radionucleotide).

In some embodiments, a linker-cytotoxic agent(s) is attached to interchain cysteine residues of an antibody or antigen-binding fragment thereof. See, e.g., WO2004/010957 and WO2005/081711. In such embodiments, the linker typically comprises a maleimide group for attachment to the cysteine residues of an interchain disulfide. In some embodiments, the linker or linker-cytotoxic agent is attached to cysteine residues of an antibody or antigen binding portion thereof as described in U.S. Pat. Nos. 7,585,491 or 8,080,250. The drug loading of the resulting conjugate typically ranges from 1 to 8.

In some embodiments, the linker or linker-cytotoxic agent is attached to lysine or cysteine residues of an antibody or antigen binding portion thereof as described in WO2005/037992 or WO2010/141566. The drug loading of the resulting conjugate typically ranges from 1 to 8.

In some embodiments, engineered cysteine residues, poly-histidine sequences, glycoengineering tags, or transglutaminase recognition sequences can be used for site-specific attachment of linkers or linker-cytotoxic agent(s) to antibodies or antigen binding portions thereof.

In some embodiments, a linker-cytotoxic agent(s) is attached to an engineered cysteine residue at an Fc region residue other than an interchain disulfide. In some embodiments, a linker-cytotoxic agent(s) is attached to an engineered cysteine introduced into an IgG (typically an IgG1) at position 118, 221, 224, 227, 228, 230, 231, 223, 233, 234, 235, 236, 237, 238, 239, 240, 241, 243, 244, 245, 247, 249, 250, 258, 262, 263, 264, 265, 266, 267, 268, 269, 270, 271, 272, 273, 275, 276, 278, 280, 281, 283, 285, 286, 291, 292, 293, 294, 295, 296, 297, 298, 299, 300, 302, 305, 313, 318, 323, 324, 325, 327,328, 329, 330, 331, 332, 333, 335, 336, 396, and/or 428, of the heavy chain and/or to a light chain at position 106, 108, 142 (light chain), 149 (light chain), and/or position V205, according to the EU numbering of Kabat. An exemplary substitution for site specific conjugation using an engineered cysteine is S239C (see, e.g., US 20100158909; numbering of the Fc region is according to the EU index).

In some embodiments, a linker or linker-cytotoxic agent(s) is attached to one or more introduced cysteine residues of an antibody or antigen binding portion thereof as described in WO2006/034488, WO2011/156328 and/or WO2016040856.

In some embodiments, an exemplary substitution for site specific conjugation using bacterial transglutaminase is N297S or N297Q of the Fc region. In some embodiments, a linker or linker-cytotoxic agent(s) is attached to the glycan or modified glycan of an antibody or antigen binding portion or a glycoengineered antibody or antigen binding portion thereof. See, e.g., WO2017/147542, WO2020123425, WO2014/072482; WO2014/065661, WO2015/057066 and WO2016/022027.

### IV. Pharmaceutical Formulations

Other aspects of the anti-MSLN antibodies and antigen binding portions thereof or other binding agents relate to compositions comprising active ingredients (i.e., including an anti-MSLN antibody or antigen-binding portion thereof or other binding agent or conjugate thereof as described herein or a nucleic acid encoding an antibody or antigen-binding portion thereof or other binding agent as described herein). In some embodiments, the composition is a pharmaceutical composition. As used herein, the term “pharmaceutical composition” refers to the active agent in combination with a pharmaceutically acceptable carrier, diluent, or excipient accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmacological composition that contains active ingredients dissolved or dispersed therein is well understood in the art and need not be limited based on any particular formulation. Typically such compositions are prepared as injectable either as liquid solutions or suspensions; however, solid forms suitable for rehydration, or suspensions, in liquid prior to use can also be prepared. A preparation can also be emulsified or presented as a liposome composition. An anti-MSLN antibody or antigen binding portion thereof or other binding agent or conjugate thereof can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, a pharmaceutical composition can contain minor amounts of auxiliary substances such as wetting or emulsifying agents, pH buffering agents and the like which enhance or maintain the effectiveness of the active ingredient (e.g., an anti-MSLN antibody or antigen binding portion thereof). The pharmaceutical compositions as described herein can include pharmaceutically acceptable salts of the components therein. Pharmaceutically acceptable salts include the acid addition salts (formed with the free amino groups of a polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain the active ingredients (e.g., an anti-MSLN antibody and/or antigen binding portions thereof or conjugate thereof) and water, and may contain a buffer such as sodium phosphate at physiological pH value, physiological saline or both, such as phosphate-buffered saline. Still further, aqueous carriers can contain more than one butter salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent that will be effective in the treatment of a particular disorder or condition will depend on the nature of the disorder or condition, and can be determined by standard clinical techniques.

The pharmaceutical compositions described herein can be formulated for oral, topical, transdermal, inhalation, parenteral, sublingual, buccal, rectal, vaginal, and intranasal administration. The term “parenteral”, as used herein, includes subcutaneous, intravenous, intramuscular, intrasternal, and intratumoral injection or infusion techniques.

In some embodiments, pharmaceutical compositions of the disclosure are formulated in a single dose unit or in a form comprising a plurality of dosage units. Methods of preparing such dosage forms are known, or will be apparent, to those skilled in this art, for example, see Remington: The Science and Practice of Pharmacy, 20th Edition (Philadelphia College of Pharmacy and Science, 2000).

In some embodiments, a pharmaceutical composition comprising an anti-MSLN antibody or antigen-binding portion thereof or conjugate thereof as described herein or a nucleic acid encoding an anti-MSLN antibody or antigen-binding portion thereof as described herein can be a lyophilisate.

In some embodiments, a syringe comprising a therapeutically effective amount of an anti-MSLN antibody or antigen binding portion thereof or conjugate thereof, or a pharmaceutical composition described herein is provided.

### IV. Therapeutic Uses of Anti-MSLN Antibodies, Antigen Binding Portions Thereof, Binding Agents, and Conjugates

In some aspects, the anti-MSLN antibodies or antigen binding portions thereof, binding agents and conjugates as described herein can be used in a method(s) comprising administering an anti-MSLN antibody or antigen-binding portion thereof or other binding agent or conjugate as described herein to a subject in need thereof. In some embodiments, the anti-MSLN antibody or antigen binding portion thereof comprises (i) a heavy chain variable region having the amino acid sequence set forth in SEQ ID NO:1, and (ii) a light chain variable region having the amino acid sequence set forth in SEQ ID NO:2. In some embodiments, the anti-MSLN antibody or antigen binding portion thereof comprises: (i) a heavy chain variable region having the amino acid sequence set forth in SEQ ID NO:1 and (ii) a light chain variable region having the amino acid sequence set forth in SEQ ID NO:2, wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the anti-MSLN antibody or antigen binding portion thereof comprises: (i) a heavy chain variable region having the amino acid sequence set forth in SEQ ID NO:1, and (ii) a light chain variable region having the amino acid sequence set forth in SEQ ID NO:2, wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. A MSLN conjugate comprises an antibody or antigen binding portion of any of these embodiments.

In some embodiments, the subject is in need of treatment for a cancer and/or a malignancy. In some embodiments, the subject is in need of treatment for a MSLN+ cancer or a MSLN+ malignancy, such as for example, mesothelioma, lung adenocarcinoma, gastric cancer, triple negative breast cancer, pancreatic cancer, ovarian adenocarcinoma, uterine serous carcinoma, endometrial adenocarcinoma, soft tissue sarcomas, head and neck cancers, or cholangiocarcinoma. In some embodiments, the method is for treating a subject having a MSLN+ cancer or malignancy. In some embodiments, the method is for treating mesothelioma in a subject. In some embodiments, the method is for treating lung adenocarcinoma in a subject. In some embodiments, the method is for treating gastric cancer in a subject. In some embodiments, the method is for treating triple negative breast cancer in a subject. In some embodiments, the method is for treating pancreatic cancer in a subject. In some embodiments, the method is for treating ovarian adenocarcinoma in a subject. In some embodiments, the method is for treating uterine serous cancer in a subject. In some embodiments, the method is for treating endometrial adenocarcinoma in a subject. In some embodiments, the method is for treating soft tissue sarcomas in a subject. In some embodiments, the method is for treating head and neck cancers in a subject. In some embodiments, the method is for treating cholangiocarcinoma in a subject.

The methods described herein include administering a therapeutically effective amount of an anti-MSLN antibody or antigen binding portion thereof or other binding agent or conjugate to a subject having a MSLN+ cancer or malignancy. As used herein, the phrase “therapeutically effective amount”, “effective amount” or “effective dose” refers to an amount of the anti-MSLN antibody or antigen binding portion thereof or other binding agent or conjugate as described herein that provides a therapeutic benefit in the treatment of management of or prevention of relapse of a cancer or malignancy, e.g. an amount that provides a statistically significant decrease in at least one symptom, sign, or marker of a tumor or malignancy. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The terms “cancer” and “malignancy” refer to an uncontrolled growth of cells which interferes with the normal functioning of the bodily organs and systems. A cancer or malignancy may be primary or metastatic, i.e. that is it has become invasive, seeding tumor growth in tissues remote from the original tumor site. A “tumor” refers to an uncontrolled growth of cells which interferes with the normal functioning of the bodily organs and systems. A subject that has a cancer is a subject having objectively measurable cancer cells present in the subject's body included in this definition are benign tumors and malignant cancers, as well as potentially dormant tumors and micro-metastases. Cancers that migrate from their original location and seed other vital organs can eventually lead to the death of the subject through the functional deterioration of the affected organs Hematologic malignancies (hematopoietic cancers), such as leukemias and lymphomas, are able to e.g., out-compete the normal hematopoietic compartments in a subject, thereby leading to hematopoietic failure (in the form of anemia, thrombocytopenia and neutropenia) ultimately causing death.

Examples of cancers include, but are not limited to, carcinomas, lymphomas, blastomas, sarcomas, and leukemias. More particular examples of such cancers include, but are not limited to, basal cell carcinoma, biliary tract cancer, bladder cancer, bone cancer, brain and CNS cancer, breast cancer (e.g., triple negative breast cancer), cancer of the peritoneum, cervical cancer; cholangiocarcinoma, choriocarcinoma, chondrosarcoma, colon and rectum cancer (colorectal cancer), connective tissue cancer, cancer of the digestive system, endometrial cancer, esophageal cancer, eye cancer, cancer of the head and neck, gastric cancer (including gastrointestinal cancer and stomach cancer), glioblastoma (GBM), hepatic carcinoma, hepatoma, intra-epithelial neoplasm, kidney or renal cancer (e.g., clear cell cancer), larynx cancer, leukemia, liver cancer, lung cancer (e.g., small-cell lung cancer, non-small cell lung cancer, adenocarcinoma of the lung, and squamous carcinoma of the lung), lymphoma including Hodgkin's and non-Hodgkin's lymphoma, melanoma, mesothelioma, myeloma, neuroblastoma, oral cavity cancer (e.g., lip, tongue, mouth, and pharynx), ovarian cancer, pancreatic cancer, prostate cancer, retinoblastoma, rhabdomyosarcoma, cancer of the respiratory system, salivary gland carcinoma, sarcoma, skin cancer, squamous cell cancer, testicular cancer, thyroid cancer, uterine or endometrial cancer, uterine serious carcinoma, cancer of the urinary system, vulval cancer, as well as other carcinomas and sarcomas, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), Hairy cell leukemia, chronic myeloblastic leukemia, and post-transplant lymphoproliferative disorder (PTLID), as well as abnormal vascular proliferation associated with phakomatoses, edema (such as that associated with brain tumors), and Meigs' syndrome.

In some embodiments, the carcinoma is selected from a solid tumor, including but not limited to, mesothelioma, lung adenocarcinoma, gastric cancer, triple negative breast cancer, pancreatic cancer, ovarian adenocarcinoma, uterine serous carcinoma, and cholangiocarcinoma.

In some embodiments, the cancer or malignancy is MSLN-positive (MSLN+). As used herein, the terms “MSLN-positive” or “MSLN+” are used to describe a cancer cell, a cluster of cancer cells, a tumor mass, or a metastatic cell that express MSLN on the cell surface (membrane-bound MSLN). Some non-limiting examples of MSLN-positive cancers include mesothelioma, lung adenocarcinoma, gastric cancer, triple negative breast cancer, pancreatic cancer, ovarian adenocarcinoma, uterine serous carcinoma, acute myeloid leukemia and cholangiocarcinoma.

It is contemplated that the methods herein reduce tumor size or tumor burden in the subject, and/or reduce metastasis in the subject. In various embodiments, tumor size in the subject is decreased by about 25-50%, about 40-70% or about 50-90% or more. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate. e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various cancers. In addition, the methods described herein can be used to treat domesticated animals and/or pets A subject can be male or female. In certain embodiments, the subject is a human.

A subject can be one who has been previously diagnosed with or identified as suffering from a MSLN+ cancer and in need of treatment, but need not have already undergone treatment for the MSLN+ cancer. Alternatively, a subject can also be one who has not been previously diagnosed as having a MSLN+ cancer in need of treatment. A subject can be one who exhibits one or more risk factors for a condition or one or more complications related to a MSLN+ cancer or a subject who does not exhibit risk factors. A “subject in need” of treatment for a MSLN+ cancer particular can be a subject having that condition or diagnosed as having that condition. In other embodiments, a subject “at risk of developing” a condition refers to a subject diagnosed as being at risk for developing the condition (e.g., a MSLN+ cancer).

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment Beneficial or desired clinical results include, but are not limited to, reduction in MSLN+ cancer cells in the subject, alleviation of one or more symptom(s), diminishment of extent of the deficit, stabilized (i e., not worsening) state of a cancer or malignancy, delay or slowing of tumor growth and/or metastasis, and an increased lifespan as compared to that expected in the absence of treatment. As used herein, the term “administering,” refers to providing a MSLN binding antibody or antigen-binding portion thereof or other binding agent or conjugate as described herein or a nucleic acid encoding the anti-MSLN antibody or antigen-binding portion thereof or other binding agent as described herein into a subject by a method or route which results in binding to the MSLN binding antibody or antigen binding portion thereof or other binding agent or conjugate to MSLN+ cancer cells or malignant cells. Similarly, a pharmaceutical composition comprising a MSLN binding antibody or antigen-binding portion thereof or other binding agent or conjugate as described herein or a nucleic acid encoding the MSLN antibody or antigen-binding portion thereof or other binding agent as described herein disclosed herein can be administered by any appropriate route which results in an effective treatment in the subject.

The dosage ranges for a MSLN binding antibody or antigen binding portion thereof or binding agent or conjugate depend upon the potency, and encompass amounts large enough to produce the desired effect e.g., slowing of tumor growth or a reduction in tumor size. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 μg/mL and 1000 μg/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg in some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be about 2 mg/kg. In some embodiments, a dose can be about 4 mg/kg. In some embodiments, a dose can be about 5 mg/kg. In some embodiments, a dose can be about 6 mg/kg in some embodiments, a dose can be about 8 mg/kg. In some embodiments, a dose can be about 10 mg/kg. In some embodiments, a dose can be about 10 mg/kg. In some embodiments, a dose can be about 12 mg/kg. In some embodiments, a dose can be from about 100 mg/m to about 700 mg/m². In some embodiments, a dose can be about 250 mg/m². In some embodiments, a dose can be about 375 mg/m². In some embodiments, a dose can be about 400 mg/m². In some embodiments, the dose can be about 500 mg/m².

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every three weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a MSLN binding antibody or antigen binding portion thereof or other MSLN binding agent or MSLN conjugate can be administered in a unit dose. The term “unit dose” when used in reference to a pharmaceutical composition refers to physically discrete units suitable as unitary dosage for the subject, each unit containing a predetermined quantity of active material (e.g., a MSLN binding antibody or antigen binding portion thereof or conjugate), calculated to produce the desired therapeutic effect in association with the required physiologically acceptable diluent, i.e., carrier, or vehicle.

In some embodiments, a MSLN binding antibody or an antigen binding portion thereof or conjugate, or a pharmaceutical composition of any of these, is administered with an immunotherapy. As used herein, “immunotherapy” refers to therapeutic strategies designed to induce or augment the subject's own immune system to fight the cancer or malignancy. Examples of an immunotherapy include, but are not limited to, antibodies such as check point inhibitors.

In some embodiments, the immunotherapy involves administration of an immune checkpoint inhibitor. In some embodiments, the immune checkpoint inhibitor is selected from inhibitors or CTLA-4, PD-1, PD-L1, PL-L2, B7-H3, B7-H14, BMA, HVEM, TIM3, GAL9, LAG3, VISTA, KIR, 2B4, CD160, CGEN-15049, CHK1, CHK2, and A2aR. In some embodiments, the immune checkpoint inhibitors include agents that inhibit CTLA-4, PD-1, PD-L1, and the like. Suitable anti-CTLA-4 therapy agents, include, for example, anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, ipilimumab, tremelimumab, anti-CTLA-4 adnectins, anti-CTLA-4 domain antibodies, single chain anti-CTLA-4 mAbs, heavy chain anti-CTLA-4 mAbs, light chain anti-CTLA-4 mAbs, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP1212422B1. Additional anti-CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present disclosure include, for example, those disclosed in: WO 98/42752; U.S. Pat. Nos. 6,682,736 and 6,207,156; Hurwitz et al., Proc. Natl. Acad. Sci. USA, 95(17) 10067-10071 (1998); Camacho et al., J. Clin. Oncology, 22(145): Abstract No. 2505 (2004) (antibody CP-675206), Mokyr et al., Cancer Res, 58:5301-5304 (1998), U.S. Pat. Nos. 5,977,318, 6,682,736, 7,109,003, and 7,132,281.

Suitable anti-PD-1 and anti-PD-L1 therapy agents, include, for example, anti-PD-1 and anti-PD-L1 antibodies, human anti-PD-1 and anti-PD-L1 antibodies, mouse anti-PD-1 and anti-PD-L1 antibodies, mammalian anti-PD-1 and anti-PD-L1 antibodies, humanized anti-PD-1 and anti-PD-L1 antibodies, monoclonal anti-PD-1 and anti-PD-L1 antibodies, polyclonal anti-PD-1 and anti-PD-L1 antibodies, chimeric anti-PD-1 and anti-PD-L1 antibodies, anti-PD-1 adnectins and anti-PD-L1 adnectins, anti-PD-1 domain antibodies and anti-PD-L1 domain antibodies, single chain anti-PD-1 mAbs and single chain anti-PD-L1 mAbs, heavy chain anti-PD-1 mAbs and heavy chain anti-PD-L1 mAbs, and light chain anti-PD-1 mAbs and light chain anti-PD-L1 mAbs. In specific embodiments, anti-PD-1 therapy agents include nivolumab, pembrolizumab, pidilizumab, MEDI0680, and combinations thereof. In other specific embodiments, anti-PD-L1 therapy agents include atezolizumab, avelumab, BMS-936559, durvalumab (MEDI4736), MSB0010718C, and combinations thereof.

Suitable anti-PD-1 and anti-PD-L1 antibodies are also described in Topalian, et al., Immune Checkpoint Blockade: A Common Denominator Approach to Cancer Therapy, Cancer Cell 27: 450-61 (Apr. 13, 2015), incorporated herein by reference in its entirety.

In some embodiments, the immune checkpoint inhibitor is Ipilimumab (Yervoy), Nivolumab (Opdivo), Pembrolizumab (Keytruda), Atezolizumab (Tecentriq), Avelumab (Bavencio), or Durvalumab (Imfinzi).

In some embodiments, provided is a method of improving treatment outcome in a subject receiving immunotherapy. The method generally includes administering an effective amount of an immunotherapy to the subject having cancer; and administering a therapeutically effective amount of a MSLN binding agent or conjugate or a pharmaceutical composition thereof to the subject, wherein the binding agent or conjugate specifically binds to MSLN+ cancer cells; wherein the treatment outcome of the subject is improved, as compared to administration of the immunotherapy alone. In some embodiments, the binding agent or conjugate thereof comprises (i) a heavy chain variable region having the amino acid sequence set forth in SEQ ID NO:1, and (ii) a light chain variable region having the amino acid sequence set forth in SEQ ID NO:2, wherein the heavy and light chain framework regions are optionally modified with from 1 to 8 amino acid substitutions, deletions or insertions in the framework regions. In some embodiments, the binding agent or conjugate thereof comprises (i) a heavy chain variable region having the amino acid sequence set forth in SEQ ID NO:1, and (ii) a light chain variable region having the amino acid sequence set forth in SEQ ID NO:2, wherein the binding agent specifically binds to MSLN+ cancer cells. In some embodiments, the binding agent is an antibody or an antigen-binding portion thereof. In some embodiments, the binding agent is a monoclonal antibody, a Fab, a Fab′, an F(ab′), an Fv, a disulfide linked Fc, a scFv, a single domain antibody, a diabody, a bi-specific antibody, or a multi-specific antibody. In some embodiments, the binding agent is a conjugate of an anti-MSLN monoclonal antibody, a Fab, a Fab′, an F(ab′), an Fv, a disulfide linked Fc, a scFv, a single domain antibody, a diabody, a bi-specific antibody, or a multi-specific antibody.

In some embodiments, the improved treatment outcome is an objective response selected from stable disease, a partial response or a complete response as determined by standard medical criteria for the cancer being treated. In some embodiments, the improved treatment outcome is reduced tumor burden. In some embodiments, the improved treatment outcome is progression-free survival or disease-free survival.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present disclosure is further illustrated by the following embodiments which should not be construed as limiting.

1. A conjugate comprising: a binding agent comprising (i) a heavy chain variable (VH) region having the amino acid sequence set forth in SEQ ID NO:1, and (ii) a light chain variable (VL) region having the amino acid sequence set forth in SEQ ID NO:2, wherein the heavy and light chain framework regions are optionally modified with from 1 to 8 amino acid substitutions, deletions or insertions in the framework regions, wherein the binding agent specifically binds to human MSLN;
at least one linker attached to the binding agent; and
at least one cytotoxic agent attached to each linker.

2. The conjugate of embodiment 1, wherein the binding agent comprises (i) a heavy chain variable region having the amino acid sequence set forth in SEQ ID NO:1, and (ii) a light chain variable region having the amino acid sequence set forth in SEQ ID NO:2.

3. A conjugate comprising: a binding agent comprising a heavy chain variable (VH) region and a light chain variable (VL) region, wherein the VH region comprises a complementarity determining region HCDR1 sequence having the amino acid sequence set forth in SEQ ID NO:11, a HCDR2 having the amino acid sequence set forth in SEQ ID NO:12, and a HCDR3 having the amino acid sequence set forth in SEQ ID NO:13, each disposed within a heavy chain framework region; and wherein the VL region comprises a LCDR1 sequence having the amino acid sequence set forth in SEQ ID NO:14, a LCDR2 having the amino acid sequence set forth in SEQ ID NO:15, and a LCDR3 having the amino acid sequence set forth in SEQ ID NO:16, each disposed within a light chain framework region;
at least one linker attached to the binding agent, and
at least one cytotoxic agent attached to each linker.

4. The conjugate of embodiment 3, wherein the framework regions are murine framework regions.

5. The conjugate of embodiment 3, wherein the framework regions are human framework regions.

6. The conjugate of any one of embodiments 1 to 5, wherein the binding agent is an antibody or an antigen-binding portion thereof.

7. The conjugate of embodiment 6, wherein the binding agent is a monoclonal antibody, a Fab, a Fab′, an F(ab′), an Fv, a disulfide linked Fc, a scFv, a single domain antibody, a diabody, a bi-specific antibody, or a multi-specific antibody.

8. The conjugate of any of the preceding embodiment, wherein the heavy chain variable region further comprises a heavy chain constant region.

9. The conjugate of embodiment 8, wherein heavy chain constant region is of the human IgG isotype.

10. The conjugate of embodiment 9, wherein the heavy chain constant region is an IgG1 constant region.

11. The conjugate of embodiment 10, wherein the IgG1 heavy chain constant region has the amino acid sequence set forth in positions 120-449 of SEQ ID NO:3.

12. The conjugate of embodiment 9, wherein the heavy chain constant region is an IgG4 constant region.

13. The conjugate of embodiment 10, wherein the heavy chain variable and constant regions have the amino acid sequence set forth in SEQ ID NO:3.

14. The conjugate of any of the preceding embodiments, wherein the light chain variable region further comprises a light chain constant region.

15. The conjugate of embodiment 14, wherein the light chain constant region is of the kappa isotype.

16. The conjugate of embodiment 15, wherein the kappa light chain constant region has the amino acid sequence set forth in positions 107-213 of SEQ ID NO:4.

17. The conjugate of embodiment 15, wherein the light chain variable and constant regions have the amino acid sequence set forth in SEQ ID NO:4.

18. The conjugate of any of embodiments 1 to 17, wherein the linker is attached to the binding agent via an interchain disulfide residue, an engineered cysteine, a glycan or modified glycan, an N-terminal residue of the binding agent or a polyhistidine residue attached to the binding agent.

19. The conjugate of any of embodiments 1 to 18, wherein the average drug loading of the conjugate is from about 1 to about 8, about 2, about 4, about 6, about 8, about 10, about 12, about 14, about 16, about 3 to about 5, about 6 to about 8 or about 8 to about 16.

20. The conjugate of any of the preceding embodiments, wherein the binding agent is mono-specific.

21. The conjugate of any of embodiments 1 to 20, wherein the binding agent is bivalent.

22. The conjugate of any of embodiments 1 to 19, wherein the binding agent comprises a second binding domain and the binding agent is bispecific.

23. The conjugate of any of the preceding embodiments, wherein the cytotoxic agent is selected from the group consisting of an auristatin, a camptothecin and a calicheamicin.

24. The conjugate of embodiment 23, wherein the cytotoxic agent is an auristatin.

25. The conjugate of embodiment 24, wherein the cytotoxic agent is monomethyl auristatin E (MMAE).

26. The conjugate of embodiment 23, wherein the cytotoxic agent is a camptothecin.

27. The conjugate of embodiment 26, wherein the cytotoxic agent is exatecan.

28. The conjugate of embodiment 23, wherein the cytotoxic agent is a calicheamicin.

29. The conjugate of embodiment 28, wherein the cytotoxic agent is SN-38.

30. The conjugate of any of the preceding embodiments, wherein the linker is selected from the group consisting of mc-VC-PAB, CL2, CL2A and (Succinimid-3-yl-N)—(CH2)n2-C(═O)-Gly-Gly-Phe-Gly-NH—CH2═OCH2-(C═O)—.

31. The conjugate of embodiment 30, wherein the linker is mc-VC-PAB

32. The conjugate of embodiment 31, wherein the linker is attached to at least one molecule of MMAE.

33. The conjugate of embodiment 30, wherein the linker is CL2A.

34. The conjugate of embodiment 33, attached to at least one molecule of SN-38.

35. The conjugate of embodiment 30, wherein the linker is CL2.

36. The conjugate of embodiment 35, attached to at least one molecule of SN-38.

37. The conjugate of embodiment 30, wherein the linker is (Succinimid-3-yl-N)—(CH2)ₙ²-C(═° O)-Gly-Gly-Phe-Gly-NH—CH₂═OCH₂—(C═O)—.

38. The conjugate of embodiment 37, wherein the linker is attached to at least one molecule of exatecan.

39. A pharmaceutical composition comprising the conjugate of any of the preceding embodiments and a pharmaceutically acceptable carrier.

40. A nucleic acid encoding the binding agent of any of embodiments 1 to 19.

41. A vector comprising the nucleic acid of embodiment 40.

42. A cell line comprising the nucleic acid of embodiment 41.

43. A method of treating a MSLN+ cancer, comprising administering to a subject in need thereof a therapeutically effective amount of the conjugate of any of embodiments 1 to 38 or the pharmaceutical composition of embodiment 39.

44. The method of embodiment 43, wherein the MSLN+ cancer is a carcinoma or a malignancy.

45. The method of embodiment 44, wherein the MSLN+ cancer is selected from melanoma, head and neck cancer, breast cancer, mesothelioma, renal clear cell cancer, chondrosarcoma, urothelial (bladder) cancer, osteosarcoma, pancreatic cancer, and leukemia (B-ALL).

46. The method of any of embodiments 43 to 45, further comprising administering an immunotherapy to the subject.

47. The method of embodiment 46, wherein the immunotherapy comprises an immune checkpoint inhibitor.

48. The method of embodiment 47, wherein the immune checkpoint inhibitor is selected from an antibody that specifically binds to human PD-1, human PD-L1, or human CTLA4.

49. The method of embodiment 48, wherein the immune checkpoint inhibitor is pembrolizumab, nivolumab, cemiplimab or ipilimumab.

50. The method of any of embodiments 43 to 49, further comprising administering chemotherapy to the subject.

51. The method of any of embodiments 43 to 50, wherein the conjugate is administered intravenously.

52. The method of any of embodiments 43 to 51, wherein the conjugate is administered in a dose of about 0.1 mg/kg to about 10 mg/kg or from about 0.1 mg/kg to about 12 mg/kg.

53. A method of improving treatment outcome in a subject receiving immunotherapy and/or chemotherapy for a MSLN+ cancer, comprising:
administering an effective amount of an immunotherapy or chemotherapy to the subject having cancer; and
administering a therapeutically effective amount of the conjugate of any of embodiments 1 to 38 or the pharmaceutical composition of embodiment 39 to the subject;
wherein the treatment outcome of the subject is improved, as compared to administration of the immunotherapy or chemotherapy alone.

54. The method of embodiment 53, wherein the improved treatment outcome is an objective response selected from stable disease, a partial response or a complete response.

55. The method of embodiment 53, wherein the improved treatment outcome is reduced tumor burden.

56. The method of embodiment 53, wherein the improved treatment outcome is progression-free survival or disease-free survival.

57. The method of embodiment 53, wherein the immunotherapy is an immune checkpoint inhibitor.

58. The method of embodiment 57, wherein the immune checkpoint inhibitor comprises an antibody that specifically binds to human PD-1, human PD-L1, or CTLA4.

59. The method of embodiment 58, wherein the immune checkpoint inhibitor is pembrolizumab, nivolumab, cemiplimab or ipilimumab.

60. The method of any of embodiments 53 to 59, wherein the conjugate is administered intravenously.

61. The method of any of embodiments 53 to 60, wherein the conjugate is administered in a dose of about 0.1 mg/kg to about 10 mg/kg.

62. Use of the conjugate of any of embodiments 1 to 38 or the pharmaceutical composition of embodiment 39 for the treatment of MSLN+ cancer in a subject.

63. Use of the conjugate of any of embodiments 1 to 38 or the pharmaceutical composition of embodiment 39 for the treatment of MSLN+ cancer in a subject receiving immunotherapy or chemotherapy.

### EXAMPLES

### Methods and Materials

The following methods and materials were used in the following examples.

Cell Culture—Ovarian cell lines (Kuramochi, OVCAR-3, CaOV3), gastric (HGC-27, NCI-N87), breast (HCC-1806) and lung carcinoma cell lines (NCI-H226, NCI-H2052, NCI-H1435, NCI-H1781, NCI-H1975) were obtained from ATCC (Manassas, VA) and maintained in culture according to the vendor's directions.

Flow Cytometry—Cells were rinsed with PBS (phosphate buffered saline, pH 7.4) and harvested with Versene (Thermo Fisher) prior to incubation with FACs buffer (PBS+1% fetal bovine serum (FBS, source BioSun) containing test antibody or ADCs for 1 h at 4° C. Cells were rinsed and washed with FACs buffer (PBS+1% FBS) and detection was performed with anti-hIgG-AF488. Cells were analyzed on a FACs Canto 11 (BD Biosciences). Quantitative FACs (qFACs) was performed to measure the number of antigen binding sites with the DAKO QifiKit (DAKO, Carpinteria, CA), according to the manufacturer's directions.

Preparation of Conjugates—ARD110-vcMMAE (ARD110-mc-vc-PAB-MMAE) was prepared by stochastic conjugation at room temperature in sodium borate buffer, pH 7.4. Briefly, ARD110 (heavy chain=SEQ ID NO:3; light chain=SEQ ID NO:4) antibody was reduced with TCEP (tris, 2-carboxyethyl phosphine) prior to incubation with drug linker, mc-vc-pab-MMAE, at 10:1 payload:antibody ratio. Excess drug linker was removed by dialysis. Size exclusion HPLC confirmed conjugate purity (99% monomer, 1% aggregate). Drug loading as assessed by LC-MS was an average of 4. The structure of ARD110-vcMMAE is shown below.

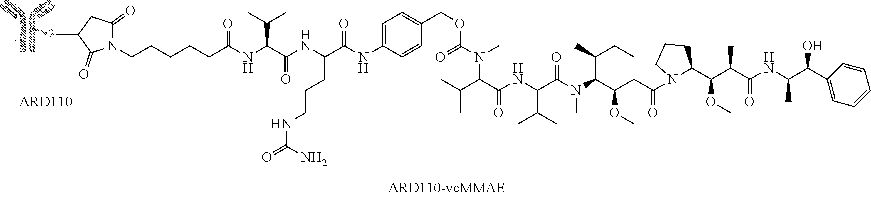 [SMILES: "ARD110" ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)C(NC(=O)C(C(C)C)N(C)C(=O)OCc1ccc(NC(=O)C(CCCNC(N)=O)NC(=O)[C@@H](NC(=O)CCCCCN2C(=O)CC(C)C2=O)C(C)C)cc1)C(C)C]

ARD110-CL2A-SN38 (ARD110-SN-38) was prepared at room temperature in PBS buffer, pH 7.4. Briefly, ARD110 antibody was reduced with TCEP prior to incubation with drug linker, CL2A-SN38, at 10:1 ratio. The reaction was stopped with N-ethylmaleimide. Excess drug linker was removed by dialysis Size exclusion HPLC confirmed conjugate purity (99% monomer, 1% aggregate). Drug loading as assesses by LC-MS was an average of 8.

In Vitro Cytotoxicity Assay—Cells were harvested with trypsin and plated in tissue culture media at 1000 to 1500 cells per well in 96-well flat clear bottom, black-walled tissue culture plates. The next day, test compounds (ADCs prepared by serial dilution to create a 10-point dose curve) or vehicle were added. The cells were incubated for 144 h. Cell viability was determined with CelltiterGlo (Promega, Madison, WI) following the manufacturer's directions. Data was graphed with Prism (GraphPad, La Jolla, CA).

Mouse Xenograft Studies—All animal experiments were conducted according to IACUC (Institutional Animal Care and Use) approved protocols following AAALAC (Association for Assessment and Accreditation of Laboratory Animal Care) guidelines. Two million HCC-1806 breast carcinoma, 10 million OVCAR3 ovarian carcinoma, 10 million NCI-H226 mesothelioma cells, or 2 million HGC-27 gastric carcinoma cells were implanted into the right flank of Balb/c mice. Tumor growth was monitored twice weekly with caliper measurements and tumor volume calculated with the formula (V=0.5a×b2 where a=longest and b=shortest diameter) Mice were treated intravenously with test compounds when tumors reached ˜150 to 300 mm³. Tumor growth, body weights, and general health of the mice were monitored for 1 to 2 weeks after the final dose of the test agent. Data was graphed with Prism (GraphPad, La Jolla, CA).

### Example 1: FACs Binding of ARD110 Antibody and Corresponding ADCs to MSLN-Positive Cell Lines

FACs binding of ARD110 antibody and corresponding ADCs to MSLN-positive cell lines was conducted. A) The cell lines HCC-1806 breast, HGC-27 and NCI-N87 gastric, CaOV3 ovarian, and NCI-12052 and NCI-H1975 lung, were incubated with 100 nM antibody, ARD110-mc-vc-PAB-MMAE ADC (ARD110-vcMMAE), ARD110-CL2A-SN-38 ADC (ARD110-SN-38) or hIgG1 isotype control for 1 hr at 4° C. Detection was done with an anti-hIgG-AF488 secondary antibody. B) HCC-1806 breast or NCI-H1975 lung carcinoma cells were incubated with increasing concentrations (8-point dose curve) of the antibody, ADC (ARD110-SN38), or hIgG1 for 1 hr at 4° C. Detection was done with an anti-hIgG-AF488 secondary antibody.

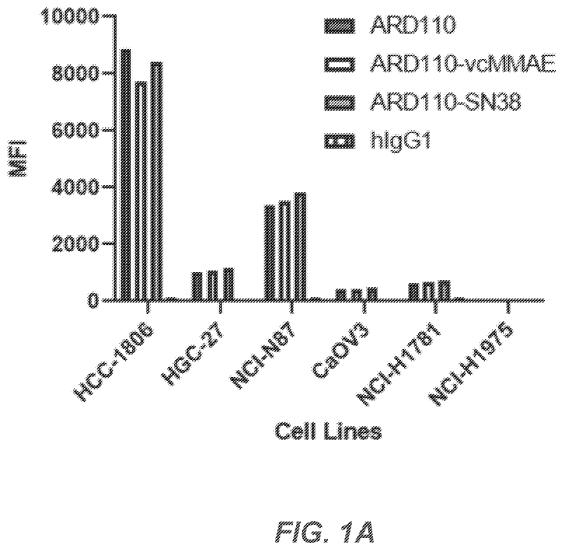

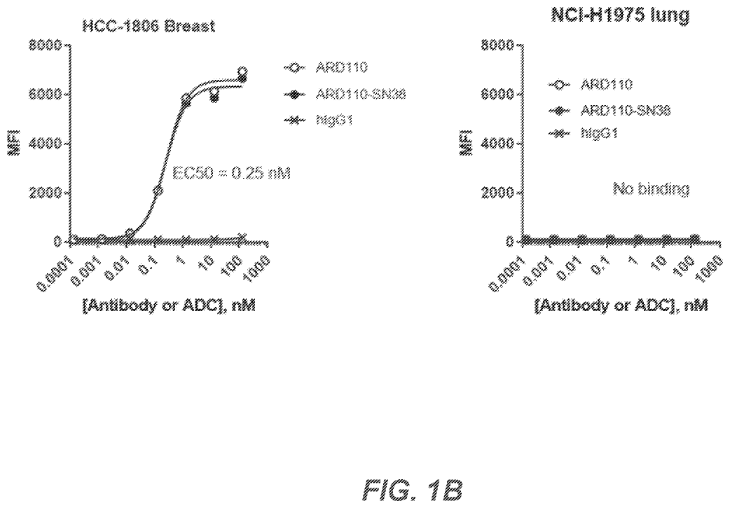

FACs binding of ARD110 antibody and corresponding ADCs to MSLN-positive cell lines are shown in FIG. 1. In FIG. 1A, there was no difference between the binding of ARD110 antibody and the ADCs for each of the cell lines tested, in particular HCC-1806 breast, HGC-27 and NCI-N87 gastric, CaOV3 ovarian, and NCI-H2052 lung. There was no binding of antibody or ADC with the NCI-H1975 lung cell line.

Similarly, the HCC-1806 breast carcinoma cell line or NCI-H1975 lung carcinoma cells was incubated with increasing concentrations (8-point dose curve) of the antibody, the ADC (ARD110-SN38), or hIgG1. The data were graphed and estimated binding constant (EC50) was determined with Prism (GraphPad Software, San Diego, CA). As can be seen in the graphs (FIG. 1B), the binding of the antibody and corresponding ADC with HCC-1806 cells was similar such that the curves overlap (estimated EC50=0.25 nM). There was no binding of antibody or ADC in the NCI-H1975 lung cell line.

### Example 2: Activities of ARD110-vcMMAE and ARD110-SN38 in an In Vitro Cytotoxicity Assay

Six human cancer cell lines (HCC-1806 breast, NCI-N87 and HGC-27 gastric, CaOV3 ovarian, and NCI-H1781 and NCI-H1975 lung) were incubated with increasing concentrations of the indicated ADCs (10-point dose curve) for 144 hrs. Cell viability was determined with CelltiterGlo.

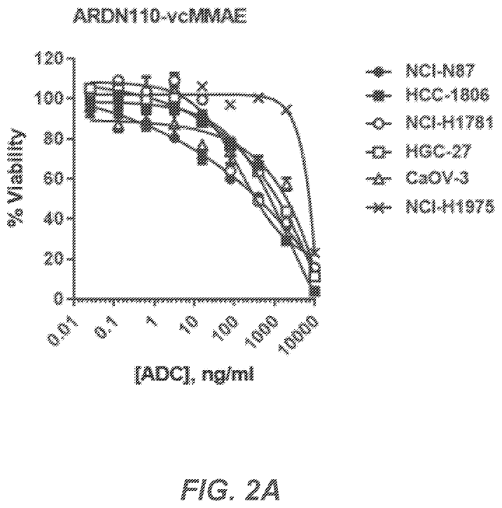

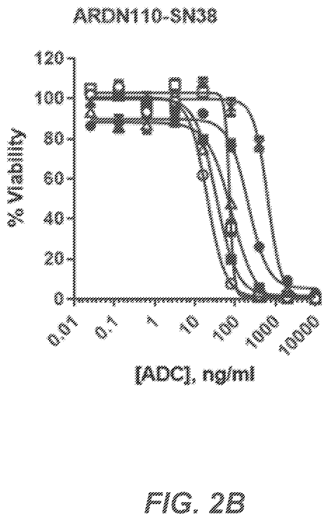

The results of the in vitro testing of ARD110-vcMMAE and ARD110-SN38 in a cytotoxicity assay are shown in FIGS. 2A and 23, respectively. ARD110-vcMMAE and ARD110-SN38 ADCs were active in inhibiting the cell growth of HCC-1806 breast, NCI-N87 and HGC-27 gastric, CaOV3 ovarian, and NCI-H1781 cells. Referring to the following table, for ARD110-SN38, the IC50s ranged from 20 ng/ml for NCI-H1781 to 213 ng/ml for NCI-N87 gastric. Under these assay conditions, ARD110-vcMMAE was less active against the same cell line panel, with higher IC50s (330 ng/ml for NCI-H1781 to 1400 ng/ml for CaOV3 ovarian). There was markedly less activity against the NCI-H1975 lung cell line with either ADC.

| | HCC- | | NCI- | NCI- | HGC- | NCI- |
|---|---|---|---|---|---|---|
| Cell Line | 1806 | CaOV3 | H1781 | N87 | 27 | H1975 |
| Indication | Breast | Ovarian | Lung | Gastric | Gastric | Lung |
| IC50 | 770 | 1400 | 330 | 365 | 1100 | 5000 |
| (ng/ml), | | | | | | |
| vcMMAE | | | | | | |
| IC50 | 34 | 83 | 20 | 213 | 73 | 700 |
| (ng/ml), | | | | | | |
| SN38 | | | | | | |

### Example 3: The Antitumor Effect of ARD110-vcMMAE and ARD110-SN38 ADCs in the OVCAR3 Ovarian Carcinoma Xenograft Model

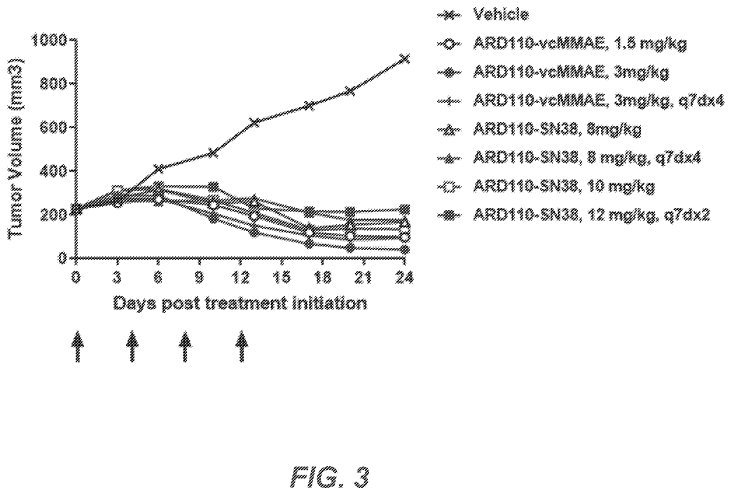

Mice were implanted with OVCAR3 ovarian cells and treated with the indicated ADCs when tumors achieved 230 mm3. ADCs were given intravenously once every 4 days for 4 doses (arrows) or as indicated in FIG. 3.

The antitumor effects of ARD110-vcMMAE and ARD110-SN38 ADCs in the OVCAR3 ovarian carcinoma xenograft model are shown in FIG. 3. ARD110-vcMMAE and ARD110-SN38 were highly effective in reducing tumor burden (p<0.001 compared to Vehicle). Treatment of mice with ARD110-vcMMAE yielded 1 complete regression when given 4 doses of 3 mg/kg.

### Example 4. The Antitumor Effect of ARD110-vcMMAE and ARD110-SN38 ADCs in the HCC-1806 Breast Carcinoma Xenograft Model

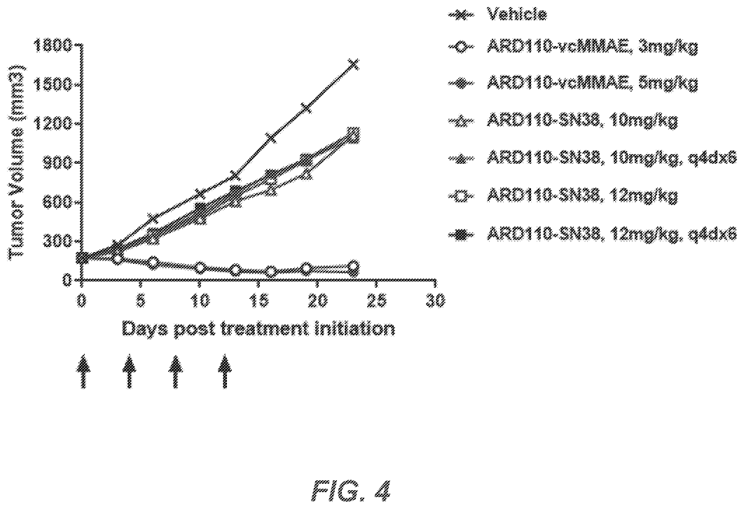

Mice were implanted with HCC-1806 breast cells and treated with the indicated ADCs when tumors achieved 176 mm³. ADCs were given intravenously once every 4 days for 4 doses (arrows) or as indicated in the legend to FIG. 4.

ARD110-vcMMAE dosed at 3 mg/kg or 5 mg/kg was highly effective in reducing tumor burden compared to Vehicle or ARD110-SN38 groups (p<0.001l). HCC-1806 is a model of TNBC. This data represents first preclinical demonstration of activity of MSLN ADC against TNBC.

### Example 5: The Antitumor Effect of ARD110-vcMMAE and ARD110-SN38 ADCs in the HGC-27 Gastric Carcinoma Xenograft Model

Mice were implanted with HGC-27 gastric cells and treated with the indicated ADCs when tumors achieved 150 mm³. ADCs were given intravenously once every 4 days for 4 doses (arrows).

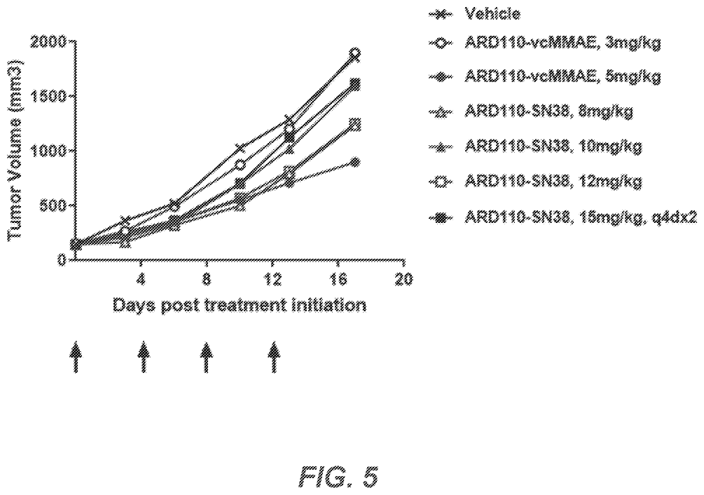

The antitumor effects of ARD110-vcMMAE and ARD110-SN38 ADCs in the HGC-27 gastric carcinoma xenograft model are shown in FIG. 5. In this fast-growing tumor model, ARD110-vcMMAE at 5 mg/kg significantly reduced tumor burden on Day 17 compared to Vehicle group (p<O 05).

### Example 6. The Antitumor Effect of ARD110-vcMMAE in the NCI-H226 Mesothelioma Xenograft Model

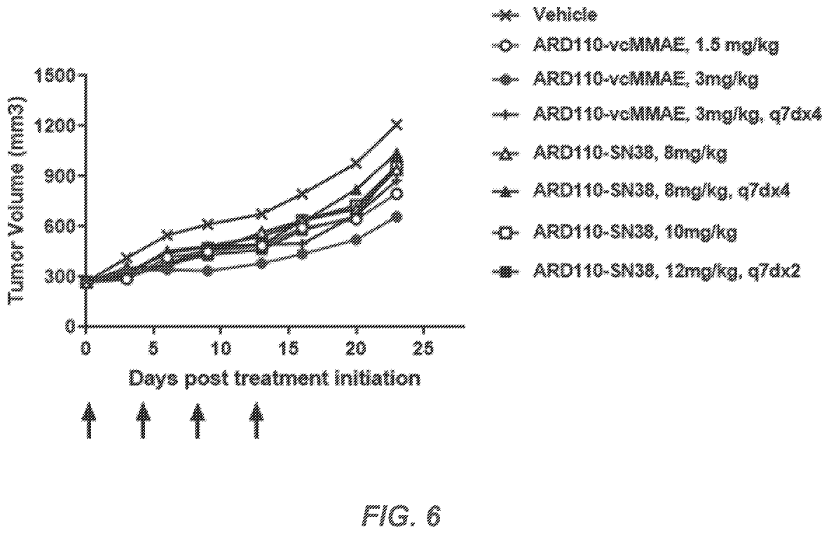

The antitumor effects of ARD110-vcMMAE and ARD110-SN38 ADCs in the NCI-H226 mesothelioma xenograft model are shown in FIG. 6. Tumor-bearing mice were given ADCs intravenously once every 4 days for 4 doses (arrows) or as indicated in FIG. 6 when tumors reached ˜300 mm³. ARD110-vcMMAE dosed at 3 mg/kg once every 4 days for 4 doses significantly reduced the large tumor burden compared to Vehicle or the ARD110-SN38 groups (p<0.025).

The present invention is not to be limited in scope by the specific embodiments described herein Indeed, various modifications of the invention in addition to those described herein will become apparent to those skilled in the art from the foregoing description and accompanying figures. Such modifications are intended to fall within the scope of the appended claims.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 63/093,254, filed on Oct. 18, 2020, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A conjugate comprising:
a binding agent comprising (i) a heavy chain variable (VH) region having the amino acid sequence set forth in SEQ ID NO:1, and (ii) a light chain variable (VL) region having the amino acid sequence set forth in SEQ ID NO:2, wherein the heavy and light chain framework regions are optionally modified with from 1 to 8 amino acid substitutions, deletions or insertions in the framework regions, wherein the binding agent specifically binds to human MSLN,
at least one linker attached to the binding agent; and
at least one cytotoxic agent attached to each linker.

2. The conjugate of claim 1, wherein the binding agent comprises: (i) a heavy chain variable region having the amino acid sequence set forth in SEQ ID NO:1, and (ii) a light chain variable region having the amino acid sequence set forth in SEQ ID NO:2.

3. A conjugate comprising:
a binding agent comprising a heavy chain variable (VH) region and a light chain variable (VL) region, wherein the VH region comprises a complementarity determining region HCDR1 sequence having the amino acid sequence set forth in SEQ ID NO:11, a HCDR2 having the amino acid sequence set forth in SEQ ID NO:12, and a HCDR3 having the amino acid sequence set forth in SEQ ID NO:13, each disposed within a heavy chain framework region; and wherein the VL region comprises a LCDR1 sequence having the amino acid sequence set forth in SEQ ID NO:14, a LCDR2 having the amino acid sequence set forth in SEQ ID NO:15, and a LCDR3 having the amino acid sequence set forth in SEQ ID NO:16, each disposed within a light chain framework region;
at least one linker attached to the binding agent; and
at least one cytotoxic agent attached to each linker.

4. The conjugate of claim 3, wherein the framework regions are murine framework regions.

5. The conjugate of claim 3, wherein the framework regions are human framework regions.

6. The conjugate of any one of claims 1 to 5, wherein the binding agent is an antibody or an antigen-binding portion thereof.

7. The conjugate of claim 6, wherein the binding agent is a monoclonal antibody, a Fab, a Fab′, an F(ab′), an Fv, a disulfide linked Fc, a scFv, a single domain antibody, a diabody, a bi-specific antibody, or a multi-specific antibody.

8. The conjugate of any one of the preceding claims, wherein the heavy chain variable region further comprises a heavy chain constant region.

9. The conjugate of claim 8, wherein heavy chain constant region is of the IgG isotype.

10. The conjugate of claim 9, wherein the heavy chain constant region is an IgG1 constant region.

11. The conjugate of claim 10, wherein the IgG1 heavy chain constant region has the amino acid sequence set forth in positions 120-449 of SEQ ID NO:3.

12. The conjugate of claim 9, wherein the heavy chain constant region is an IgG4 constant region.

13. The conjugate of claim 10 or 11, wherein the heavy chain variable and constant regions have the amino acid sequence set forth in SEQ ID NO: 3.

14. The conjugate of any of the preceding claims, wherein the light chain variable region further comprises a light chain constant region.

15. The conjugate of claim 14, wherein the light chain constant region is of the kappa isotype.

16. The conjugate of claim 15, wherein the kappa light chain constant region has the amino acid sequence set forth in positions 107-213 of SEQ ID NO:4.

17. The conjugate of claim 15 or 16, wherein the light chain variable and constant regions have the amino acid sequence set forth in SEQ ID NO:4.

18. The conjugate of any of claims 1 to 17, wherein the linker is attached to the binding agent via an interchain disulfide residue, an engineered cysteine, a glycan or modified glycan, an N-terminal residue of the binding agent or a polyhistidine residue attached to the binding agent.

19. The conjugate of any of claims 1 to 18, wherein the average drug loading of the conjugate is from about 1 to about 8, about 2, about 4, about 6, about 8, about 10, about 12, about 14, about 16, about 3 to about 5, about 6 to about 8 or about 8 to about 16.

20. The conjugate of any of the preceding claims, wherein the binding agent is mono-specific.

21. The conjugate of any of claims 1 to 20, wherein the binding agent is bivalent.

22. The conjugate of any of claims 1 to 19, wherein the binding agent comprises a second binding domain and the binding agent is bispecific.

23. The conjugate of any of the preceding claims, wherein the cytotoxic agent is selected from the group consisting of an auristatin, a camptothecin and a calicheamicin.

24. The conjugate of claim 23, wherein the cytotoxic agent is an auristatin.

25. The conjugate of claim 24, wherein the cytotoxic agent is MMAE.

26. The conjugate of claim 23, wherein the cytotoxic agent is a camptothecin.

27. The conjugate of claim 26, wherein the cytotoxic agent is exatecan.

28. The conjugate of claim 23, wherein the cytotoxic agent is a calicheamicin.

29. The conjugate of claim 28, wherein the cytotoxic agent is SN-38.

30. The conjugate of any of the preceding claims, wherein the linker is selected from the group consisting of mc-VC-PAB, CL2, CL2A and (Succinimid-3-yl-N)—(CH2)ₙ²—C(═O)-Gly-Gly-Phe-Gly-NH—CH₂—OCH₂-(C═O)—.

31. The conjugate of claim 30, wherein the linker is mc-VC-PAB.

32. The conjugate of claim 31, wherein the linker is attached to at least one molecule of MMAE.

33. The conjugate of claim 30, wherein the linker is CL2A.

34. The conjugate of claim 33, attached to at least one molecule of SN-38.

35. The conjugate of claim 30, wherein the linker is CL2.

36. The conjugate of claim 35, attached to at least one molecule of SN-38.

37. The conjugate of claim 30, wherein the linker is (Succinimid-3-yl-N)—(CH₂)ₙ²—C(═O)-Gly-Gly-Phe-Gly-NH—CH₂—O—CH₂—(C═O)—.

38. The conjugate of claim 37, wherein the linker is attached to at least one molecule of exatecan.

39. A pharmaceutical composition comprising the conjugate of any of the preceding claims and a pharmaceutically acceptable carrier.

40. A nucleic acid encoding the binding agent of any of claims 1 to 19.

41. A vector comprising the nucleic acid of claim 40.

42. A cell line comprising the nucleic acid of claim 41.

43. A method of treating a MSLN+ cancer, comprising administering to a subject in need thereof a therapeutically effective amount of the conjugate of any of claims 1 to 38 or the pharmaceutical composition of claim 39.

44. The method of claim 43, wherein the MSLN+ cancer is a carcinoma or a malignancy.

45. The method of claim 44, wherein the MSLN+ cancer is selected from mesothelioma, lung adenocarcinoma, gastric cancer, triple negative breast cancer, pancreatic cancer, ovarian adenocarcinoma, uterine serous carcinoma, endometrial adenocarcinoma, soft tissue sarcomas, head and neck cancers or cholangiocarcinoma.

46. The method of any of claims 43 to 45, further comprising administering an immunotherapy to the subject.

47. The method of claim 46, wherein the immunotherapy comprises a checkpoint inhibitor.

48. The method of claim 47, wherein the checkpoint inhibitor is selected from an antibody that specifically binds to human PD-1, human PD-L1, or human CTLA4.

49. The method of claim 48, wherein the checkpoint inhibitor is pembrolizumab, nivolumab, cemiplimab or ipilimumab.

50. The method of any of claims 43 to 49, further comprising administering chemotherapy to the subject.

51. The method of any of claims 43 to 50, wherein the conjugate is administered intravenously.

52. The method of any of claims 43 to 51, wherein the conjugate is administered in a dose of about 0.1 mg/kg to about 12 mg/kg.

53. A method of improving treatment outcome in a subject receiving immunotherapy and/or chemotherapy for a MSLN+ cancer, comprising:
administering an effective amount of an immunotherapy or chemotherapy to the subject having cancer; and
administering a therapeutically effective amount of the conjugate of any of claims 1 to 38 or the pharmaceutical composition of claim 39 to the subject,
wherein the treatment outcome of the subject is improved, as compared to administration of the immunotherapy or chemotherapy alone.

54. The method of claim 53, wherein the improved treatment outcome is an objective response selected from stable disease, a partial response or a complete response.

55. The method of claim 53, wherein the improved treatment outcome is reduced tumor burden.

56. The method of claim 53, wherein the improved treatment outcome is progression-free survival or disease-free survival.

57. The method of claim 53, wherein the immunotherapy is an immune checkpoint inhibitor.

58. The method of claim 57, wherein the immune checkpoint inhibitor comprises an antibody that specifically binds to human PD-1, human PD-L1, or CTLA4.

59. The method of claim 58, wherein the immune checkpoint inhibitor is pembrolizumab, nivolumab, cemiplimab or ipilimumab.

60. The method of any of claims 53 to 59, wherein the conjugate is administered intravenously.

61. The method of any of claims 53 to 60, wherein the conjugate is administered in a dose of about 0.1 mg/kg to about 10 mg/kg.

62. Use of the conjugate of any of claims 1 to 38 or the pharmaceutical composition of claim 39 for the treatment of MSLN+ cancer in a subject.

63. Use of the conjugate of any of claims 1 to 38 or the pharmaceutical composition of claim 39 for the treatment of MSLN+ cancer in a subject receiving immunotherapy or chemotherapy.


SEQUENCE LISTING (22 sequences)

SEQ ID NO: 1 - protein, 119 aa, Artificial Sequence
  Synthetic sequence ARD110 VH
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 2 - protein, 106 aa, Artificial Sequence
  Synthetic sequence ARD110 VL
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 3 - protein, 449 aa, Artificial Sequence
  Synthetic sequence ARD110 VH-IgG1
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 213 aa, Artificial Sequence
  Synthetic sequence ARD110 VL-Igkappa
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 19 aa, Artificial Sequence
  VH Signal Sequence
MEFGLSWLFLVAILKGVQC

SEQ ID NO: 6 - protein, 22 aa, Artificial Sequence
  VL Signal Sequence
MDMRVPAQLLGLLLLWFPGSRC

SEQ ID NO: 7 - protein, 468 aa, Artificial Sequence
  ARD110 VH-IgG1 with signal sequence
MEFGLSWLFLVAILKGVQCQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 235 aa, Artificial Sequence
  ARD110 VL-Igkappa with signal sequence
MDMRVPAQLLGLLLLWFPGSRCDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 622 aa, Artificial Sequence
  Synthetic sequence MSLN isoform 1
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 10 - protein, 630 aa, Artificial Sequence
  Synthetic sequence MSLN isoform 2
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 11 - protein, 5 aa, Artificial Sequence
  Synthetic sequence ARD110 VH CDR1
GYTMN

SEQ ID NO: 12 - protein, 17 aa, Artificial Sequence
  Synthetic sequence ARD110 VH CDR2
LITPYNGASSYNQKFRG

SEQ ID NO: 13 - protein, 10 aa, Artificial Sequence
  Synthetic sequence ARD110 VH CDR3
GGYDGRGFDY

SEQ ID NO: 14 - protein, 10 aa, Artificial Sequence
  Synthetic sequence ARD110 VL CDR1
SASSSVSYMH

SEQ ID NO: 15 - protein, 7 aa, Artificial Sequence
  Synthetic sequence ARD110 VL CDR2
DTSKLAS

SEQ ID NO: 16 - protein, 9 aa, Artificial Sequence
  Synthetic sequence ARD110 VL CDR3
QQWSKHPLT

SEQ ID NO: 17 - protein, 25 aa, Artificial Sequence
  Synthetic  linker sequence
  VARIANT (6)..(25): amino acids 6-25, 11-25, 16-25, or 21-25 may be present or absent
GGGGSGGGGSGGGGSGGGGSGGGGS

SEQ ID NO: 18 - protein, 6 aa, Artificial Sequence
  Synthetic sequence hexaHtidinyl tag
HHHHHH

SEQ ID NO: 19 - DNA, 1350 bp, Artificial Sequence
  Synthetic sequence ARD110 Coding Sequence for VH and IgG1
caggtacaactgcagcagtctggccctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggatccgggaccccggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 20 - DNA, 643 bp, Artificial Sequence
  Synthetic sequence Coding Sequence for ARD110 VL and Igkappa
gacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtaagcaccctctcacgttcggatccgggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaat

SEQ ID NO: 21 - DNA, 357 bp, Artificial Sequence
  Synthetic sequence ARD110 VH Coding Region
caggtacaactgcagcagtctggccctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggatccgggaccccggtcaccgtctcctca

SEQ ID NO: 22 - DNA, 318 bp, Artificial Sequence
  Synthetic sequence ARD110 VL Coding Region
gacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtaagcaccctctcacgttcggatccgggaccaaggtggaaatcaaa